SEQUENCE LISTING (14 sequences)

SEQ ID NO: 1 - RNA, 64 nt, Artificial
  siRNA targeting human DNA polymerase beta mRNA
gauccccaugcugcagaugcaggauauucaagagauauccugcaucugcagcauuuuuuggaaa

SEQ ID NO: 2 - RNA, 64 nt, Artificial
  siRNA targeting human DNA polymerase beta mRNA
gauccccgaucaguacuacugugguguucaagagacaccacaguaguacugaucuuuuuggaaa

SEQ ID NO: 3 - RNA, 64 nt, Artificial
  siRNA targeting human DNA polymerase beta mRNA
gauccccuugcuacagucuguggcaguucaagagacugccacagacuguagcaauuuuuggaaa

SEQ ID NO: 4 - RNA, 64 nt, Artificial
  siRNA targeting human DNA polymerase beta mRNA
gauccccugaguacaccauccgucccuucaagagagggacggaugguguacucauuuuuggaaa

SEQ ID NO: 5 - RNA, 59 nt, Artificial
  siRNA targeting human DNA polymerase beta mRNA
cccaaggaccggagcgaaugaggccuguauccucccuggcagacacaacccaauaggag

SEQ ID NO: 6 - RNA, 50 nt, Artificial
  siRNA targeting human DNA polymerase beta mRNA
ccaucccagcuucacuucagaaucaaccaaacagccaaaacuguuacauc

SEQ ID NO: 7 - RNA, 65 nt, Artificial
  siRNA targeting human DNA polymerase beta mRNA
ugaaaaagaauauccacacagaagaauugauaucagguugauacccaaagaucaguauuacugug

SEQ ID NO: 8 - DNA, 25 bp, Artificial
  oligonucleotide primer
caccatgagcaaacgcaaggcgccg

SEQ ID NO: 9 - DNA, 21 bp, Artificial
  oligonucleotide primer
tcattcacttctatccttggg

SEQ ID NO: 10 - DNA, 19 bp, Artificial
  oligonucleotide
atgctgcagatgcaggata

SEQ ID NO: 11 - DNA, 19 bp, Artificial
  oligonucleotide
gatcagtactactgtggtg

SEQ ID NO: 12 - DNA, 21 bp, Artificial
  oligonucleotide
cctgccctgagcngctgtggg

SEQ ID NO: 13 - DNA, 9 bp, Artificial
  cDNA encoding hairpin bulge for siRNA
ttcaagaga

SEQ ID NO: 14 - DNA, 19 bp, Artificial
  cDNA encoding target sequence for siRNA targeting human DNA polymerase beta mRNA
gaagaacgtgagccaagct

(19) United States
(12) Patent Application Publication
(10) Pub. No.: US 2010/0048682 A1
(43) Pub. Date: Feb. 25, 2010

(54) INHIBITING DNA POLYMERASE BETA TO ENHANCE EFFICACY OF ANTICANCER AGENTS

(75) Inventor: Robert W. Sobol, Pittsburgh, PA (US)

Correspondence Address: KLARQUIST SPARKMAN, LLP, 121 SW SALMON STREET, SUITE 1600, PORTLAND, OR, 97204, US

(73) Assignee: UNIVERSITY OF PITTSBURGH - OF THE COMMONWEALTH SYSTEM OF HIGHER EDUCATION

(21) Appl. No.: 12/605,254

(22) Filed: Oct. 23, 2009

Related U.S. Application Data
(63) Continuation of application No. 11/913,288, filed on Jun. 20, 2008.
(60) Provisional application No. 60/682,696, filed on May 19, 2005.

(51) Int. Cl. A61K 31/7088; C12N 5/02

(52) U.S. Cl. 514/44 R; 435/375; 514/44 A

(57) ABSTRACT

The invention provides anticancer methods. In one embodiment, the inventive method involves the co-administration to cancerous cells of (a) a chemotherapeutic agent, radiation, or a combination of a chemotherapeutic agent and radiation and (b) an inhibitor of DNA polymerase beta. In another embodiment, the invention provides anticancer methods involving the co-administration to cancerous cells of (a) a chemotherapeutic agent, radiation, or a combination of a chemotherapeutic agent and radiation and (b) an siRNA or shRNA in an amount sufficient to attenuate base excision repair within the cell. Another aspect of the invention relates to pharmaceutical compositions comprising an siRNA or shRNA that attenuates base excision repair.

### CROSS-REFERENCE TO RELATED APPLICATIONS

This is a continuation of U.S. patent application Ser. No. 11/913,288, filed on Oct. 31, 2007, which is the U.S. National Stage of International Application No. PCT/US2006/019606, filed May 19, 2006, which was published in English under PCT Article 21(2), which in turn claims the benefit of U.S. provisional application 60/682,696, filed on May 19, 2005. The prior applications are incorporated herein by reference in their entirety.

### FIELD OF THE INVENTION

The invention relates to anticancer methods and compositions.

### BACKGROUND OF THE INVENTION

DNA alkylating agents have a central role in the curative therapy of many human tumors, yet resistance to these agents limits their effectiveness. The efficacy of the alkylating agent temozolomide (TMZ) has been attributed to the induction of O6-MeG, a DNA lesion repaired by the protein MGMT. Resistance to TMZ has been ascribed to elevated levels of MGMT and/or reduced mismatch repair. However, a need remains for enhancing the efficacy of anticancer agents such as TMZ and other alkylating agents.

### BRIEF SUMMARY OF THE INVENTION

The invention provides anticancer methods. In one embodiment, the inventive method involves the co-administration to cancerous cells of (a) a chemotherapeutic agent, radiation, or a combination of a chemotherapeutic agent and radiation and (b) an inhibitor of DNA polymerase beta. In another embodiment, the invention provides anticancer methods involving the co-administration to cancerous cells of (a) a chemotherapeutic agent, radiation, or a combination of a chemotherapeutic agent and radiation and (b) an siRNA or shRNA in an amount sufficient to attenuate base excision repair within the cell. In a preferred embodiment, a chemotherapeutic agent is an alkylating agent, such as TMZ.

Another aspect of the invention relates to pharmaceutical compositions comprising an siRNA or shRNA that attenuates base excision repair.

These aspects, and additional inventive features, will be apparent from reviewing the attached figures and the following detailed description.

### BRIEF DESCRIPTION OF THE SEVERAL VIEWS OF THE DRAWINGS

FIG. 1a-b: Mouse and Human Beta pol siRNA target design. FIG. 1a depicts the sequence of the murine mBETA-502 oligo, which was found not to induce knockdown. The first underlined region corresponds to 5′-3′ sense mBeta mRNA and the second underlined region corresponds to 5′-3′ antisense mBeta mRNA. The first 7 base pairs at the 5′ end represent the BglII site overhang. The final 3 base pairs at the 3′ end represent the HindIII site overhang. The 7 base pairs just before the HindIII site represent the t-tail. The hairpin bulge is located between the 2 underlined regions (base pairs ttcaagaga) (SEQ ID NO:13). FIG. 1a also depicts the sequence of the murine mBETA-826 oligo, which was found to induce strong knockdown. The first underlined region corresponds to 5′-3′ sense mBeta mRNA and the second underlined region corresponds to 5′-3′ antisense mBeta mRNA. The first 7 base pairs at the 5′ end represent the BglII site overhang. The final 3 base pairs at the 3′ end represent the HindIII site overhang. The 7 base pairs just before the HindIII site represent the t-tail. The hairpin bulge is located between the 2 underlined regions (base pairs ttcaagaga) (SEQ ID NO:13). By using new design software, 5 potential beta pol targets of 19 base pairs each were identified starting at base pair 77, 80, 157, 743, and 744, respectively. FIG. 1b depicts the sequence of the human hBpol-521 oligo, which was found not to induce knockdown. The first underlined region corresponds to 5′-3′ sense hBeta mRNA and the second underlined region corresponds to 5′-3′ antisense mBeta mRNA. The first 7 base pairs at the 5′ end represent the BglII site overhang. The final 3 base pairs at the 3′ end represent the HindIII site overhang. The 7 base pairs just before the HindIII site represent the t-tail. The hairpin bulge is located between the 2 underlined regions (base pairs ttcaagaga) (SEQ ID NO:13). FIG. 1b also depicts the sequence of the human hBpol-882 oligo, which was found not to induce knockdown. The first underlined region corresponds to 5′-3′ sense hBeta mRNA and the second underlined region corresponds to 5′-3′ antisense mBeta mRNA. The first 7 base pairs at the 5′ end represent the BglII site overhang. The final 3 base pairs at the 3′ end represent the HindIII site overhang. The 7 base pairs just before the HindIII site represent the t-tail. The hairpin bulge is located between the 2 underlined regions (base pairs ttcaagaga) (SEQ ID NO:13). Using new design software, it was discovered that the human beta pol target is gaagaacgtgagccaagct (SEQ ID NO:14), which corresponds to base pairs 78-96 (19 base pairs). The software also suggested 5 other potential targets of 19 base pairs in length, beginning at base pair 398, 399, 402, 405, and 839, respectively.

FIG. 2a-b. TMZ methylation damage is repaired by the pol-β dependent BER pathway. Cell survival following TMZ treatment. Cells were cultured for 24 hr, treated with TMZ for 2 hr and viable cells were measured after 48 hr by a modified MTT assay (MTS). (a) Wt cells (filled circles), Aag null cells (filled diamonds), pol-β null cells (filled squares) and pol-β null/Aag null cells (filled triangle); (b) Wt cells (filled circles), pol-β null cells (filled squares), pol-λ, null cells (filled diamond) and pol-ι null cells (filled triangle). Means are calculated from quadruplicate values in each experiment. Results indicate the mean±S.D. of four independent experiments.

FIG. 3a-d. siRNA mediates long-term pol-β knockdown and induces a TMZ hypersensitive phenotype. (a) Ectopic expression of V5-pol-β in 293T cells (transfected pVS.mpolB, lanes 1-3) and its inhibition by co-transfected pol-β siRNA (pSuper.mpol-β787/805, lane 1 or pSuper.mpol-β463/481, lane 2) as analyzed by immunoblotting using anti-V5. α-Tubulin blot is included as loading control, (b) siRNA down-regulation of endogenous pol-β protein expression in Wt cells (92TAg) transfected with an siRNA-expressing plasmid (pSuper.mpol-β787/805). Stable clones were selected as described in Materials and Methods. 20 μg nuclear extract was prepared from Wt cells transfected with a control pSuper vector (lane 1), Wt cells expressing pSuper.mpol-β787/805 (wtpolβ-KD.2, wt polβ-KD.3 & wt polβ-KD.4; lanes 2-4), pol-β null cells (lane 5) and parental Wt cells (lane 6) and analyzed for pol-β expression by immunoblot (upper panel). Blots were re-probed for the expression of PCNA as a loading control. Cells were cultured for 24 hr and (c) cells were treated with TMZ for 2 hr and viable cells were measured after 48 hr or (d) cells were treated with TMZ for 48 hr and viable cells were measured immediately. Viability was determined by a modified MTT assay (MTS). Wt cells (filled circles), pol-β null cells (filled squares), Wt cells expressing control siRNA (WtCont, open circles), Wt cells expressing pol-β specific siRNA (wtpolβ-KD.2, clone 2, filled triangle; wt polβ-KD.3, clone 3, filled diamond and wt polβ-KD.4, clone 4, inverted filled triangle). Means are calculated from quadruplicate values in each experiment. Results indicate the mean±S.D. of four independent experiments.

FIG. 4a-d. Over-expression of human Aag in pol-β down-regulated cells results in increased sensitivity of TMZ. Cell survival following TMZ treatment. Cells were cultured for 24 hr and (a,c) cells were treated with TMZ for 2 hr and viable cells were measured after 48 hr or (b,d) cells were treated with TMZ for 48 hr and viable cells were measured immediately. Viability was determined by a modified MTT assay (MTS). (a,b): Wt cells (filled circles), pol-β null cells (filled squares), Wt cells expressing hAag (WthAag.3, clone 3, open triangle; WthAag.8, clone 8, inverted open triangle); (c,d): Wt cells (filled circles), pol-β null cells (filled squares), Wt cells expressing pol-fc specific siRNA (wt polβ-KD.4, clone 4, inverted filled triangle), Wt cells expressing pol-fc specific siRNA and hAag (wt hAag.2/polβ-KD.4, clone 2, lower shaded squares; wt hAag.3/polβ-KD.4, clone 3, upper shaded squares). Means are calculated from quadruplicate values in each experiment. Results indicate the mean±S.D. of four independent experiments.

FIG. 5. Deletion of pol-β results in increased expression of γ-H2AX. Phosphorylation of H2AX following treatment with MMS and TMZ in Wt and pol-β null cells. γ-H2AX expression following increasing concentrations of MMS (top panel) and TMZ (bottom panel) in Wt and pol-β null cells relative to PCNA as a loading control. γ-H2AX expression was quantified using Quantity One analysis software and a Bio-Rad chemi-doc Imager and represented as the fold of control.

FIG. 6a-d: Increased temozolomide sensitivity in Human Cancer cells after pol-β knockdown mediated by expression of pol-β specific siRNA. FIG. 6a depicts the sequence alignment of 3 siRNA sequences specific to human pol-β, the human pol-β ORF and the Genebank sequence NM002690. FIG. 6b presents data demonstrating the levels of pol-β expression in nuclear proteins isolated from the breast cancer cell line MDA-MB-231. A western blot analysis was utilized to show the expression of pol-β in control MDA-MB-231 breast cancer cells (CTL) and lack of expression of pol-β on three MDA-MB-231 breast cancer cell clones after stable expression of the pol-β specific siRNA lentivirus. PCNA expression is the same in all, shown as a loading control. FIG. 6c presents data demonstrating that decreasing the expression of pol-β leads to an increased cellular sensitivity to temozolomide. Cells (as labeled) were cultured in 96-well plates for 24 hours prior to exposure to TMZ for 48 hours. After exposure, cells were washed, re-fed growth medium and viable cells were determined using a modified MTT assay. Plots show the % viable cells as compared to untreated cells. FIG. 6d depicts the target sites for each of 5 pol-β specific shRNA vectors.

FIG. 7: DNA pol-β expression as determined by immunoblot analysis of nuclear proteins isolated from the breast cancer cell line MDA-MB-231 or MDA-MB-231 cells transduced with a human pol-β shRNA Lentiviral vector. Proteins isolated from three separate shRNA-expressing clones are shown (lanes 2-4) as compared to proteins isolated from control cells (lane 1). MPG and APE1 expression determined by immunoblot. PCNA expression is shown as a loading control (lower panel).

FIG. 8a: Down-regulation of endogenous pol-β protein expression in MDA-MB-231 cells induces a TMZ hypersensitive phenotype. MDA-MB-231 cells or MDA-MB-231 cells expressing pol-β specific shRNA (as labeled) were cultured in 96-well plates for 24 hours prior to exposure to TMZ. Viable cells were determined using a modified MTT assay. Plots show the % viable cells as compared to untreated (Control) cells. Means are calculated from quadruplicate values in each experiment. Results indicate the mean±S.E. of four independent experiments.

FIG. 8b: (Panel A) Growth of parental MDA-MB-231 cells and several derived clones expressing pol-β specific shRNA. Tumors were measured each week after implantation of 1×10⁷ cells. (Panel B) DNA pol-β expression as determined by immunoblot analysis of nuclear proteins isolated from the tumor xenografts. Lane 1—breast cancer cell line MDA-MB-231; Lane 2—MDA-MB-231 cells that harbor the human pol-β shRNA Lentiviral vector. (Panel C,D) Immunhistochemical analysis of 4 week old xenografts using an affinity-purified pol-β polyclonal Ab. Panel C=MDA-MB-231 xenografts; Panel D=Xenograft from MDA-MB-231 cells that harbor the human pol-β shRNA Lentiviral vector.

FIG. 9: MPG over-expression as determined by immunoblot analysis of nuclear proteins isolated from the MDA-MB-231 or MPG over-expressing MDA-MB-231 cells. Proteins isolated from three separate MPG over-expressing clones are shown (lanes 2-4) as compared to proteins isolated from control cells (lane 1). Pol-β and APE1 expression determined by immunoblot. PCNA expression is shown as a loading control (lower panel).

FIG. 10b: Over-expression of human MPG in MDA-MB-231 cells shifts the rate-limiting step in the BER pathway and induces a TMZ hypersensitive phenotype. Cells (as labeled) were cultured in 96-well plates for 24 hours prior to exposure to TMZ. Viable cells were determined using a modified MTT assay as described in FIG. 8a.

FIG. 10a: Cells (as labeled) were cultured in 96-well plates for 24 hours prior to exposure to TMZ. Viable cells were determined 48 hours post treatment using a modified MTT assay. Plots show the % viable cells as compared to untreated (Control) cells.

FIG. 11: MPG over-expression as determined by immunoblot analysis of nuclear proteins isolated from the MDA-MB-231 or MPG over-expressing MDA-MB-231 cells transduced with a human pol-β shRNA Lentiviral vector. Proteins isolated from two separate MPG over-expressing clones are shown (lanes 3-4) as compared to proteins isolated from control cells (lane 1) and human pol-β knockdown(KD) cells (lane 2). pol-β and APE1 expression determined by immunoblot. PCNA expression is shown as a loading control (lower panel).

FIG. 12: Over-expression of human MPG in pol-β down-regulated MDA-MB-231 cells results in further increase in sensitivity to TMZ. Cells (as labeled) were cultured in 96-well plates for 24 hours prior to exposure to TMZ. Viable cells were determined using a modified MTT assay as described in FIG. 8a.

FIG. 13: DNA pol-β expression as determined by immunoblot analysis of nuclear proteins isolated from the MDA-MB-231 or pol-β KD/MDA-MB-231 cells transfected with the human Flag-pol-β. Proteins isolated from three separate Flag-pol-β expressing clones are shown (lanes 4-6) as compared to proteins isolated from control cells (lane 1), pol-β KD/MDA-MB-231 cells (lane 2) or pIRES-Neo transfected pol-β KD/MDA-MB-231 cells (lane 3). Flag, MPG and APE1 expression determined by immunoblot. PCNA expression is shown as a loading control (lower panel). For immunoprecipitation (IP), cell lysate from the above cell lines was incubated overnight with anti-Flag antibodies followed by 1-h incubation with Protein G Dynabeads. Immunoprecipitates were eluted with 3× Flag peptide, separated on SDS-PAGE and transferred to nitrocellulose filters and probed with anti-pol-β antibody.

FIG. 14: Reconstitution of Flag-pol-β in pol-β down-regulated MDA-MB-231 cells restores resistance to TMZ. Cells (as labeled) were cultured in 96-well plates for 24 hours prior to exposure to TMZ. Viable cells were determined using a modified MTT assay as described in FIG. 8a.

FIG. 15: DNA pol-β expression as determined by immunoblot analysis of nuclear proteins isolated from the MDA-MB-231 or pol-β KD/MDA-MB-231 cells transfected with the polymerase inactive mutant human Flag-pol-β (D256A). Proteins isolated from three separate Flag-pol-β (D256A) expressing clones are shown (lanes 4-6) as compared to proteins isolated from control cells (lane 1), pol-β KD/MDA-MB-231 cells (lane 2) or pIRES-Neo transfected pol-β KD/MDA-MB-231 cells (lane 3). Flag, MPG and APE1 expression determined by immunoblot. PCNA expression is shown as a loading control (lower panel). For immunoprecipitation (IP), cell lysate from the above cell lines was incubated overnight with anti-Flag antibodies followed by 1-h incubation with Protein G Dynabeads. Immunoprecipitates were eluted with 3× Flag peptide, separated on SDS-PAGE and transferred to nitrocellulose filters and probed with anti-pol-β antibody.

FIG. 16: Reconstitution of polymerase inactive mutant human Flag-pol-β (D256A) in pol-β down-regulated MDA-MB-231 cells restores resistance to TMZ. Cells (as labeled) were cultured in 96-well plates for 24 hours prior to exposure to TMZ. Viable cells were determined using a modified MTT assay as described in FIG. 8a.

FIG. 17: DNA pol-β expression as determined by immunoblot analysis of nuclear proteins isolated from the MDA-MB-231 or MDA-MB-231 cells transfected with human MPG. Proteins isolated from three separate MPG over-expressing clones are shown (lanes 4-6) as compared to proteins isolated from control cells (lane 1), MPG over-expressing/MDA-MB-231 cells (lane 2) or pIRES-Puro transfected in MPG over-expressing/MDA-MB-231 cells (lane 3). Flag, MPG and APE1 expression determined by immunoblot. PCNA expression is shown as a loading control (lower panel). For immunoprecipitation(IP), cell lysate from the above cell lines was incubated overnight with anti-Flag antibodies followed by 1-h incubation with Protein G Dynabeads. Immunoprecipitates were eluted with 3× Flag peptide, separated on SDS-PAGE and transferred to nitrocellulose filters and probed with anti-pol-β antibody.

FIG. 18: Reconstitution of Flag-pol-β in human MPG over-expressing MDA-MB-231 cells restores resistance to TMZ. Cells (as labeled) were cultured in 96-well plates for 24 hours prior to exposure to TMZ. Viable cells were determined using a modified MTT assay as described in FIG. 2.

### DETAILED DESCRIPTION OF THE INVENTION

In one aspect, the invention relates to the use of (a) a chemotherapeutic agent, radiation, or a combination of a chemotherapeutic agent and radiation and (b) an inhibitor of DNA polymerase beta to prepare medicaments to be used adjunctively to kill or retard the growth of cancers. In this respect, the invention provides a method of killing or retarding the proliferation of one or more neoplastic or cancerous cells involving (a) administering a chemotherapeutic agent, radiation, or a combination of a chemotherapeutic agent and radiation to the cell(s) and (b) administering an inhibitor of DNA polymerase beta to the cell(s). The inhibitor of DNA polymerase beta is administered in an amount sufficient to reduce the activity of DNA polymerase beta within the cell, which potentates the activity of the chemotherapeutic agent, radionuclide or combination thereof. In an alternative embodiment, step (b) instead involves administering a small interfering RNA (“siRNA” or “shRNA”) that targets an enzyme that facilitates base excision repair within the cell(s). In this embodiment, the siRNA or shRNA is administered to the cell in an amount to attenuate base excision repair, which potentiates the activity of the chemotherapeutic agent, radiation, or combination thereof.

In accordance with the inventive method, the chemotherapeutic agent, radiation, or combination thereof can be administered prior to or following the administration of the inhibitor of DNA polymerase beta or the siRNA or shRNA that targets an enzyme that facilitates base excision repair; alternatively, agents (a) and (b) can be administered concurrently.

In the context of the inventive method, the cell can be separated but typically is within a population of neoplastic or cancerous cells. The cell can be in vitro, in which the method can be used for research. However, preferably, the cell is in vivo, in which instance the method facilitates a method of treating a cancer patient in need of such treatment. While preferably such patient is human, the method also is applicable to veterinary application. Moreover, the method is broadly applicable to many types of cancers, such as skin cancers (e.g., melanoma, keratocarcinoma, etc.) brain cancers (e.g., glioblastoma), cancers of the gastrointestinal tract (e.g., throat cancer, esophageal cancer, stomach cancer, intestinal cancer, colon cancer, colorectal cancer), cancers of the lungs, breast cancers, liver cancers, pancreatic cancers, ovarian cancers, testicular cancers, prostate cancers, lymphomas, and other cancers.

For in vivo application, the chemotherapeutic agent, radiation, or a combination thereof, as well as the inhibitor of DNA polymerase beta or the siRNA or shRNA that targets an enzyme that facilitates base excision repair, are administered to the patient in an amount and at a location sufficient to contact one or more cancerous cell(s) within the patient. Such route of administration can be any method or route commonly employed to administer such anticancer agents to patients, which are well known to those of ordinary skill in the art. For example, such agents can be administered systemically, topically, transdermally, orally, or by intravenous, intraatrial, peritoneal, or intratumoral injection. Similarly, the inhibitor of DNA polymerase beta or the siRNA or shRNA that targets an enzyme that facilitates base excision repair can be suitably formulated and administered to patients in accordance with standard methods to achieve delivery of the inhibitor of DNA polymerase beta to the cells within the patient.

The neoplastic or cancerous cell within the patient can be an isolated cell, such as metastatic cells in circulation. Alternatively, the cell(s) can be within a tumor within the patient. In this embodiment, the inventive method can be employed to retard the growth of a tumor within a patient having a tumor. It will be noted that in accordance with this embodiment, the chemotherapeutic agent, radiation, or a combination thereof, as well as the inhibitor of DNA polymerase beta or the siRNA or shRNA that targets an enzyme that facilitates base excision repair, are administered to the patient in an amount and at a location sufficient to contact one or more cancerous cell(s) within the tumor. By killing or retarding the proliferation of cells within the tumor, the inventive method thereby retards the growth of the tumor. In preferred embodiments, the tumor is shrunk or eliminated as a result of the inventive method.

As noted, in some embodiments, the chemotherapeutic agent, radiation, or combination thereof, as well as the inhibitor of DNA polymerase beta (or the siRNA or shRNA that targets an enzyme that facilitates base excision repair) can be co-administered. While such agents can be formulated by known methodologies, to facilitate the inventive method, the invention also provides a pharmaceutical composition comprising siRNA or shRNA as an active agent and either a chemotherapeutic agent, radionuclide, or combination of chemotherapeutic agent and radionuclide as a second active agent, and a pharmaceutically acceptable carrier. The siRNA or shRNA can target an enzyme involved in DNA base excision repair so as to attenuate base excision repair within cells, and preferably the siRNA or shRNA targets DNA polymerase beta mRNA.

The inventive composition can be formulated for delivery of the agents by any desired route, such as systemically, topically, transdermally, orally, or by intravenous, intraatrial, peritoneal, or intratumoral injection. Typically, such pharmaceutical compositions will contain from about 0.1% to about 95% by weight of the active agent(s); preferably, from about 5% to about 70% by weight; and more preferably from about 10% to about 60% by weight of the active agent(s).

Any conventional carrier or excipient may be used in the pharmaceutical compositions of the invention. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially-available from, for example, Sigma, P.O. Box 14508, St. Louis, Mo. 63178. By way of further illustration, conventional formulation techniques are described in Remington: The Science and Practice of Pharmacy, 20.sup.th Edition, Lippincott Williams & White, Baltimore, Md. (2000); and H. C. Ansel et al., Pharmaceutical Dosage Forms and Drug Delivery Systems, 7.sup.th Edition, Lippincott Williams & White, Baltimore, Md. (1999).

Representative examples of materials which can serve as pharmaceutically acceptable carriers include, but are not limited to, the following: (1) sugars, such as lactose, glucose and sucrose; (2) starches, such as corn starch and potato starch; (3) cellulose, such as microcrystalline cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The pharmaceutical compositions of the invention are typically prepared by thoroughly and intimately mixing or blending a compound of the invention with a pharmaceutically-acceptable carrier and one or more optional ingredients. If necessary or desired, the resulting uniformly blended mixture can then be shaped or loaded into tablets, capsules, pills and the like using conventional procedures and equipment.

The pharmaceutical compositions of the invention are preferably packaged in a unit dosage form. The term “unit dosage form” refers to a physically discrete unit suitable for dosing a patient, i.e., each unit containing a predetermined quantity of active agent calculated to produce the desired therapeutic effect either alone or in combination with one or more additional units. For example, such unit dosage forms may be capsules, tablets, pills, and the like.

In one embodiment, the pharmaceutical compositions of the invention are suitable for injection (e.g. parenteral, intravenous, intratumoral, etc.). Such compositions can be formulated by admizixing the active agents with a suitable volume of water for injection, and desired buffers.

In another embodiment, the pharmaceutical compositions of the invention are suitable for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present invention as an active ingredient.

When intended for oral administration in a solid dosage form (i.e., as capsules, tablets, pills and the like), the pharmaceutical compositions of the invention will typically comprise a compound of the present invention as the active ingredient and one or more pharmaceutically-acceptable carriers, such as sodium citrate or dicalcium phosphate. Optionally or alternatively, such solid dosage forms may also comprise: (1) fillers or extenders, such as starches, microcrystalline cellulose, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and/or sodium carbonate; (5) solution retarding agents, such as paraffin; (6) absorption accelerators, such as quaternary ammonium compounds; (7) wetting agents, such as cetyl alcohol and/or glycerol monostearate; (8) absorbents, such as kaolin and/or bentonite clay; (9) lubricants, such as talc, calcium stearate, magnesium stearate, solid polyethylene glycols, sodium lauryl sulfate, and/or mixtures thereof; (10) coloring agents; and (11) buffering agents.

Release agents, wetting agents, coating agents, sweetening, flavoring and perfuming agents, preservatives and antioxidants can also be present in the pharmaceutical compositions of the invention. Examples of pharmaceutically-acceptable antioxidants include: (1) water-soluble antioxidants, such as ascorbic acid, cysteine hydrochloride, sodium bisulfate, sodium metabisulfite sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like. Coating agents for tablets, capsules, pills and like, include those used for enteric coatings, such as cellulose acetate phthalate (CAP), polyvinyl acetate phthalate (PVAP), hydroxypropyl methylcellulose phthalate, methacrylic acid-methacrylic acid ester copolymers, cellulose acetate trimellitate (CAT), carboxymethyl ethyl cellulose (CMEC), hydroxypropyl methyl cellulose acetate succinate (HPMCAS), and the like.

If desired, the pharmaceutical compositions of the present invention may also be formulated to provide slow or controlled release of the active ingredient using, by way of example, hydroxypropyl methyl cellulose in varying proportions; or other polymer matrices, liposomes and/or microspheres.

In addition, the pharmaceutical compositions of the present invention may optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Suitable liquid dosage forms for oral administration include, by way of illustration, pharmaceutically-acceptable emulsions, microemulsions, solutions, suspensions, syrups and elixirs. Such liquid dosage forms typically comprise the active ingredient and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active ingredient, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Alternatively, the pharmaceutical compositions of the invention are formulated for administration by inhalation. Suitable pharmaceutical compositions for administration by inhalation will typically be in the form of an aerosol or a powder. Such compositions are generally administered using well-known delivery devices, such as a metered-dose inhaler, a dry powder inhaler, a nebulizer or a similar delivery device.

When administered by inhalation using a pressurized container, the pharmaceutical compositions of the invention will typically comprise the active ingredient and a suitable propellant, such as dichlorodifluoromethane, trichlorofluoromethane, dichlorotetrafluoroethane, carbon dioxide or other suitable gas.

Additionally, the pharmaceutical composition may be in the form of a capsule or cartridge (made, for example, from gelatin) comprising a compound of the invention and a powder suitable for use in a powder inhaler. Suitable powder bases include, by way of example, lactose or starch.

The compounds of the invention can also be administered transdermally using known transdermal delivery systems and excipients. For example, a compound of the invention can be admixed with permeation enhancers, such as propylene glycol, polyethylene glycol monolaurate, azacycloalkan-2-ones and the like, and incorporated into a patch or similar delivery system. Additional excipients including gelling agents, emulsifiers and buffers, may be used in such transdermal compositions if desired.

In the context of the foregoing description of the inventive method and composition, where a chemotherapeutic agent is employed, preferably it is an alkylator, such as nitrosourea and most preferably TMZ. However, other agents can be used (e.g., 3-bis(2-chloroethyl)-1-nitrosourea (BCNU), 1-(4-amino-2-methyl-5-pyrimidinyl)methyl-3-(2-chloroethyl)-3-nitrosourea (ACNU), 1-(2-Chloroethyl)-3-cyclohexyl-1-nitrosourea (CCNU), Dacarbazine (DTIC), sarmustine, chlorambucil, or melphalan).

Many inhibitors of DNA polymerase beta suitable for use in the context of the inventive method and compositions are known in the art (see, e.g., Chaturvedula et al., J. Nat. Prod. May, 67(5), 899-901, 2004; Chaturvedula et al., J. Nat. Prod., 66, 1463-1465, 2003; Chaturvedula et al., Bioorg Med. Chem., 12(23), 6271-75, 2004; Deng et al., J. Nat. Prod., 62(12), 1624-26, 1999; Chaturvedula et al., J. Nat. Prod., 67(6), 964-67, 2004; and Deng et al., J. Nat. Prod., 63(10):1356-60, 2000). Non-limiting examples include an acylated oleanane triterpenoid from Couepia polyandra (e.g., 3beta, 16beta, 23-triacetoxyolean-12-en-28-oic acid); an ursane triterpene from Monochaetum vulcanicum (e.g., 3beta-acetoxy-2alpha-hydroxyurs-12-en-28-oic acid); a 7,8-euphadien-type triterpenoid from Brackenridgea nitida and Bleasdalea bleasdalei (e.g., (24E)-3beta-hydroxy-7,24-euphadien-26-oic acid); a harbinatic acid from Hardwickia binata (3alpha-O-trans-p-coumaroyl-7-labden-15-oic acid); a lupane triterpenoid from Solidago canadensis (3beta-(3R-acetoxyhexadecanoyloxy)-lup-20(29)-ene, 3beta-(3-ketohexadecanoyloxy)-lup-20(29)-ene, 3beta-(3R-acetoxyhexadecanoyloxy)-29-nor-lupan-20-one, or 3beta-(3-hetohexadecanoyloxy)-29-nor-lupan-20-one); a neolignan from Endlicheria aff (e.g., (7S,8R,1′S,5′S,6′R)-Delta(2′,8′)-5′,6′-dihydroxy-3′-methoxy-3,4-methylenedioxy-4′-oxo-8.1′,7,5′-neolignan, (7S,8R,1′S,5′S,6′R)-Delta(2′,8′)-3′,5′,6′-trihydroxy-3,4-methylenedioxy-4′-oxo-8.1′,7,5′-neolignan, 2,4-dimethoxy-5,6-methylenedioxy-1-(2-propenyl)benzene, 2,6-dimethoxy-3,4-methylenedioxycinnamyl alcohol); a pentacyclic triterpenoid from Freziera; a triterpenoid from Baeckea gunniana (e.g., 3 beta-hydroxyrus-12,19(29)-dien-28-oic acid or 3 beta-hydroxyrus-18,20(30)-dien-28-oic acid); a bis-5-alkylresorcinol from Panopsis rubescens (e.g., 1,3-dihydroxy-5-[14′-(3″,5″-dihydroxyphenyl)-cis-4′-tetradecenyl]benzene, 1,3-dihydroxy-5-[14′-(3″ ″, 5′-dihydroxyphenyl)-cis-7′-tetradecenyl]benzene, or 1,3-dihydroxy-5-[14′-(3∂ ″,5″-dihydroxyphenyl)tetradecenyl]benzene); (7S,8R,1′S,5′S,6′R)-Delta(2′,8′)-3′,6′-dihydroxy-5′-methoxy-3,4-methylenedioxy-4′-oxo-8.1′,7,5′-neolignan; 1-allyl-2,6-dimethoxy-3,4-methylenedioxybenzene; 2alpha-hydroxyursolic acid; 3-(p-coumaroyl) ursolic acid; 3′-methoxyguianin; alpha-amyrin acetate; armenin-B; beta-sitosterol; beta-sitosteryl-beta-d-galactoside; betulinic acid; canellin A; canellin C; cycloartenol; cycloartenol palmitate; dillapiole; lupeol; lupeyl acetate; Myristinin A; oleanolic acid; omega-hydroxyisodillapiole; stigmasterol; and ursolic acid.

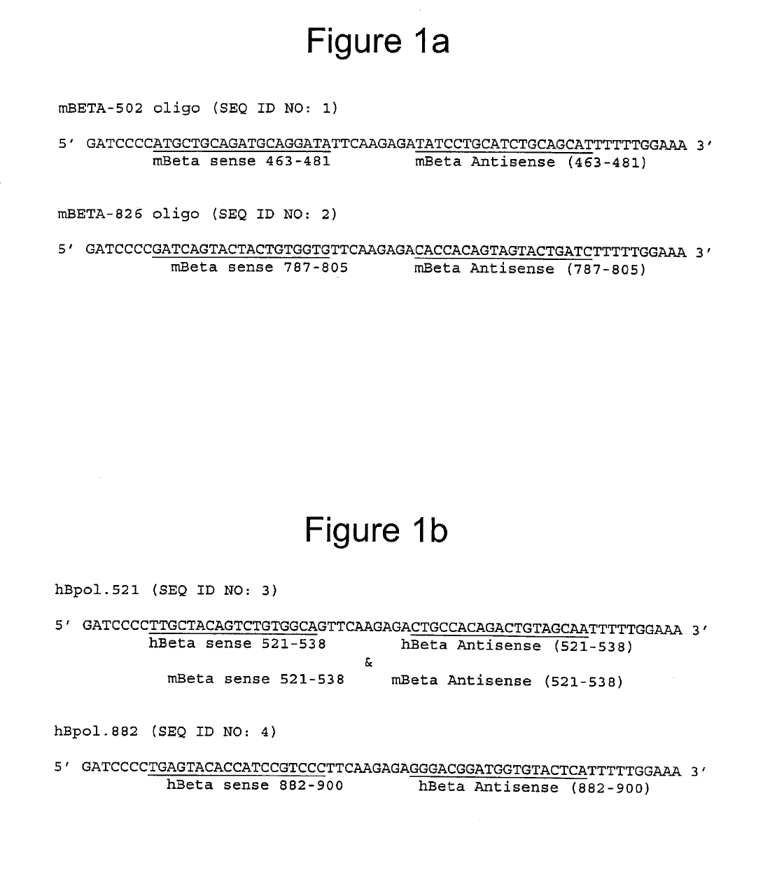

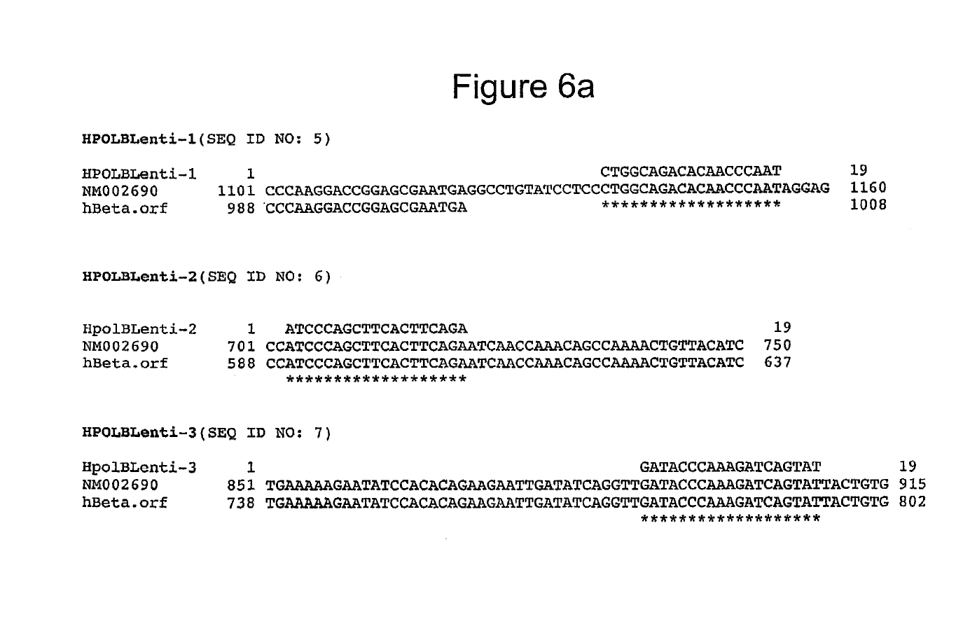

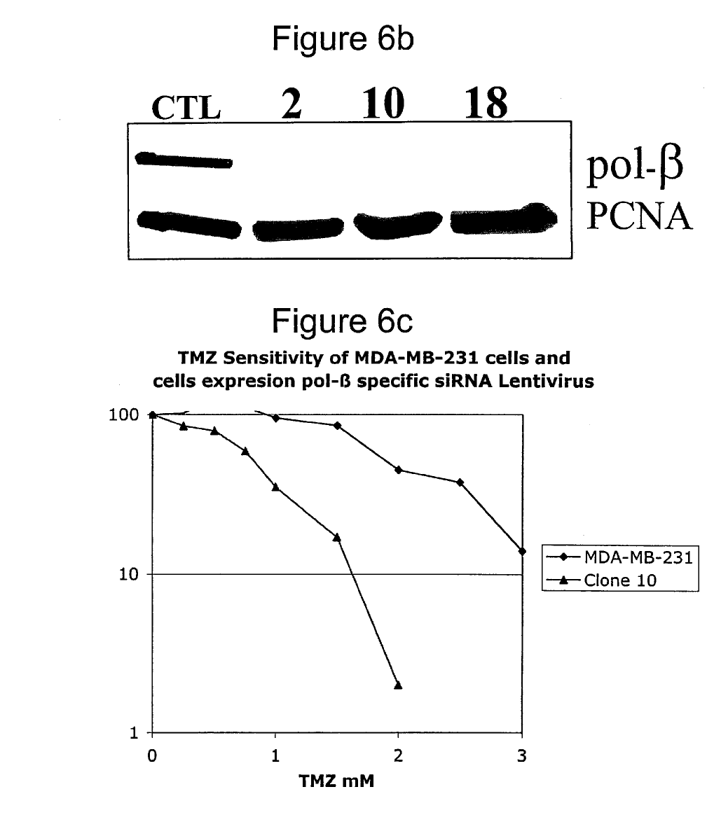

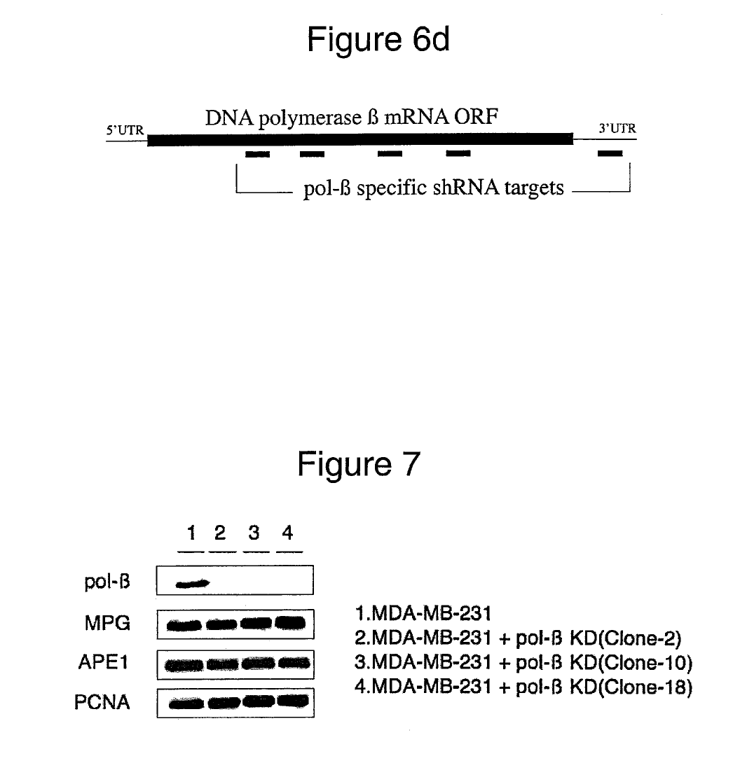

In other embodiments, the activity of DNA polymerase beta is inhibited by RNA interference, in which case the inhibitor of DNA polymerase beta that can be employed in the inventive method and composition can include a small interfering RNA (“siRNA” or “shRNA”) that targets the mRNA of an enzyme that facilitates base excision repair (such as DNA polymerase beta) within the cancerous cells. As the DNA polymerase beta mRNA sequence is known (GenBank Accession No. NM_{—}002690), suitable siRNA or shRNA species can be constructed by known methods to have a region complementary to the DNA polymerase beta mRNA sense and antisense sequence (including all 5′ and 3′ UTR sequences), separated by a spacer region, which facilitates for formation of hairpins (see, e.g., FIGS. 1a and 1b and 6a). It will be observed that siRNA or shRNA species for inhibiting DNA polymerase beta are known in the art (see, e.g., Polosina et al., DNA Repair, 3, 1469-74 (2004)). Other non-limiting examples of suitable RNAs for RNA interference of DNA polymerase beta include:

(SEQ ID NO: 1)

gauccccaugcugcagaugcaggauauucaagagauauccugcaucugca
gcauuuuuuggaaa;

(SEQ ID NO: 2)

gauccccgaucaguacuacugugguguucaagagacaccacaguaguacu
gaucuuuuuggaaa;

(SEQ ID NO: 3)

gauccccuugcuacagucuguggcaguucaagagacugccacagacugua
gcaauuuuuggaaa;

(SEQ ID NO: 4)

gauccccugaguacaccauccgucccuucaagagagggacggauggugua
cucauuuuuggaaa;

(SEQ ID NO: 5)

cccaaggaccggagcgaaugaggccuguauccucccuggcagacacaacc
caauaggag;

(SEQ ID NO: 6)

ccaucccagcuucacuucagaaucaaccaaacagccaaaacuguuacau
c;

(SEQ ID NO: 7)

ugaaaaagaauauccacacagaagaauugauaucagguugauacccaaag
aucaguauuacugug

### Example 1

This example demonstrates that siRNA or shRNA is effective as a long-term down-regulator of pol-β and that this down-regulation leads to an increased sensitivity to TMZ.

### Materials and Methods

Chemicals and Reagents. Cell culture supplies were from InVitrogen-Gibco. TMZ was from the National Cancer Institute Developmental Therapeutics Program and prepared as a 100 mM stock in DMSO. Methyl methanesulfonate (MMS) and Mitomycin C (MMC) were purchased from Sigma-Aldrich (St. Louis, Mo.). The following primary antibodies were employed: anti-pol-β (Mab clone 18S), a kind gift from S. H. Wilson, NIEHS, NIH; anti-hAag, provided by T. R. O'Connor, City of Hope National Medical Center, Duarte, Calif.; anti-V5 (InVitrogen); anti-γ-H2AX (Upstate Biotechnology); anti-α-tubulin (Oncogene Research Products) and anti-PCNA (Santa Cruz). All electrophoresis reagents were from Bio-Rad. Neomycin and Puromycin were purchased from Invitrogen and BD Clontech, respectively.

Plasmid Expression Vectors and RNAi Development. The following mammalian expression vectors were employed: murine V5-polβ, pV5.mpolβ; human Aag, pRS1422; mpolβ specific siRNA expression plasmids, pSuper.mpol-β463/481 and pSuper.mpol-β787/805. A murine pol-β N-terminal V5-fusion mammalian expression plasmid (pV5S.mpol β) was constructed as follows: Total RNA was isolated from primary mouse embryonic fibroblasts (MEFs), cDNA was prepared (InVitrogen; Superscript) and the murine pol-β cDNA was PCR amplified using primers mbetaF (caccatgagcaaacgcaaggcgccg (SEQ ID NO:8)) and mbetaR (tcattcacttctatccttggg (SEQ ID NO:9)). The PCR amplification product was cloned into the pENTR-TOPO plasmid using the directional TOPO cloning method (InVitrogen) to yield pENTR.mpolβ. The sequence of the cloned cDNA was then confirmed by the UPCI sequencing core facility. pV5.mpolβ was then developed from pENTR.mpolβ by lambda phage mediated site-specific recombination with pcDNA3.1/nV5-DEST (Gateway, InVitrogen). The mpolβ specific siRNA expression plasmids were developed using the algorithm for siRNA/shRNA design from Oligoengine. Oligonucleotides were designed to target murine pol-β mRNA (mpol-β 463/481; sequence 5′-atgctgcagatgcaggata-3′ (SEQ ID NO:10) and mpol-β 787/805; sequence 5′-gatcagtactactgtggtg-3′ (SEQ ID NO:11)) and cloned into the pSUPER vector (Oligoengine) within the BglII/HindIII restriction sites, yielding pSuper.mpol-β 463/481 and pSuper.mpol-β 787/805.

Cell Lines and Transfection Conditions. Transformed MEF cell lines (92TAg, Wt; 88TAg, pol-β null; 308TAg, Aag null and 283TAg, pol-β null/Aag null) have been described previously and are available from the ATCC. 293T cells were a gift from J. O'Bryan (NIEHS, NIH). Primary cultures of pol-λ, null MEFs were a kind gift from C.A. Reynaud (Faculte de Medecine Necker-Enfants Malades, Paris, France). The pol-ι null MEFs were derived from C129SvJ mice as described previously and identified by PCR to confirm the pol-ι null mutation. These were immortalized by SV40 large T-antigen (370TAg, pol-λ, null; 369TAg, pol-ι null) as described previously. Human Aag over-expressing cell lines were prepared as follows: briefly, 1.5×10⁵ cells were seeded into 60 mm dishes and incubated for 24-30 hours at 10% CO₂ at 37° C. The Aag expression plasmid (pRS1422) was transfected using FuGene 6 Transfection Reagent (Roche Diagnostic Corp) according to the manufacturer's instructions. Stable cell lines were selected in G418 (600 μg/ml) for 2 weeks, individual clones were amplified and 20 μg of nuclear extract was analyzed by immunoblotting for the expression of human Aag protein and then re-probed for expression of pol-β and PCNA.

Transfection of the pSuper siRNA plasmids was completed as follows: Briefly, 1.5×10⁵ cells were seeded into 60 mm dishes and incubated for 24-30 hours at 10% CO₂ at 37° C. Plasmids were transfected using FuGene 6 Transfection Reagent (Roche Diagnostic Corp) according to the manufacturer's instructions. For transient transfections, cells were lysed 24 hours post transfection by boiling in Laemmli buffer and analyzed by immunoblotting for the expression of the V5-pol-β transgene using anti-V5 (Invitrogen). Stable cell lines were isolated following transfection as above followed by selection in puromycin (7.5 μg/ml) for 2 weeks. Individual clones were amplified and 20 μg of nuclear extract was analyzed by immunoblotting for the expression of endogenous pol-β protein using the Nucbuster nuclear protein extract reagent (Novagen) and then re-probed for expression of PCNA as a loading control.

Culture Conditions and Cell Cytotoxicity Assays. Transformed MEFs were cultured at 37° C. in a humidified incubator with 10% CO₂ in DMEM supplemented with 10% fetal bovine serum, penicillin (50 units/ml), streptomycin (50 μg/ml) and Glutamax (4 mM). Human embryonic kidney cells (293T) were cultured similarly in DMEM supplemented with 10% fetal bovine serum, penicillin (50 units/ml), streptomycin (50 μg/ml) and Glutamine (4 mM). TMZ, MMS and MMC induced cytotoxicity was determined by growth inhibition assays. Briefly, cells were seeded in 96-well dishes at 1250 cells/well. After 24 hours, cells (quadruplicate wells) were exposed to serial dilutions of damaging agent in growth media for 1-2 hours (or as indicated in the text) at 37° C. Drug-containing medium was replaced with fresh medium and the plates were incubated at 37° C. for 48 hours at which point the total cell number was determined by a modified MTT assay (MTS; Promega). Metabolically active cells were quantified by the bioreduction of the MTS tetrazolium compound by recording absorbance at 490 nm using a microplate reader. Results were calculated from the average of four separate experiments and are reported as the % of treated cells relative to the cells in control wells (% Control).

Cell Extract Preparation and Immunoblot Assays. Nuclear extracts were prepared using the NucBuster nuclear protein extract reagent (Novagen, Madison, Wis.). Protein concentration was determined by Bio-Rad protein assay reagents, according to the manufacturer's instruction. Nuclear protein (20 μg) was separated by electrophoresis in a 10% SDS-polyacrylamide gel and electro-transferred to a 0.45 μM nitrocellulose membrane (Trans-Blot, Bio-Rad). Membranes were blocked by overnight incubation in a 5% dried milk/TBS solution at 4° C. Antigens of interest (i.e., pol-β, AAG, V5) were detected by incubating the membrane for 2 hours at room temperature with the primary antibody. The membrane was washed with TBST (10 mM Tris-HCI, pH 8, 150 mM NaCI and 0.05% Tween 20) and incubated with horseradish peroxidase (HRP)-conjugated secondary antibody (goat anti-mouse HRP; Bio-Rad) at room temperature for 1 hour. HRP activity was detected by enhanced chemiluminescence (Bio-Rad). Each membrane was stripped and re-probed with anti-α-tubulin or anti-PCNA antibodies to correct for differences in protein loading.

For γ-H2AX immunoblotting, protein was prepared as follows: briefly, 6×10⁵ cells were seeded into 150 mm dishes and incubated for 24 hours at 10% CO₂ at 37° C. Cells were treated with MMS and TMZ for 1 or 2 hr respectively, and whole cells were scraped from the plates in 1×PBS, washed, and immediately placed on dry ice. Thawed pellets were re-suspended in a 1:1 ratio of RIPA buffer and Laemmli buffer. Samples were boiled for 5 minutes and protein from an equal cell number was added to each well. Protein was separated by electrophoresis in a 12% SDS-polyacrylamide gel and electro-transferred to a 0.45 μM nitrocellulose membrane (Trans-Blot, Bio-Rad). Membranes were blocked for 20 minutes in a 3% dried milk/TBS solution at room temperature and incubated with 0.5-1 μg/ml of anti-phospho-H2AX (Serine 139) (Upstate) prepared in TBS/3% dried milk and 0.1% Tween20 over night at 4° C. Membranes were washed twice with water and incubated with horseradish peroxidase (HRP)-conjugated secondary antibody (goat anti-rabbit HRP; Bio-Rad) at room temperature for 1.5 hour. Following a series of washes with water and TBS, HRP activity was detected using the Super Signal West Femto reagent (Pierce). Each membrane was re-probed with anti-PCNA to correct for differences in protein loading, and densitometry was used to quantitate with Quantity One 1-D analysis software (BioRad).

Alkyladenine DNA glycosylase Activity Assay. Aag activity was measured using a double-stranded oligonucleotide (21-bp) substrate containing a single etheno-adenine (eA) lesion: 5′-cctgccctgagceAgctgtggg-3′ (SEQ ID NO:12) (Trevigen, Gaithersburg, Md.), as described previously. 20 μg whole cell protein extract was incubated with ³²P-5′-labeled substrate (0.5 pmol) for 0, 15, 30, 45 and 60 minutes at 37° C. and product was analyzed by electrophoretic separation on 16% polyacrylamide gel (7M urea, TBE). The reaction product was visualized by autoradiography and quantified by phosphorimager analysis.

### Results

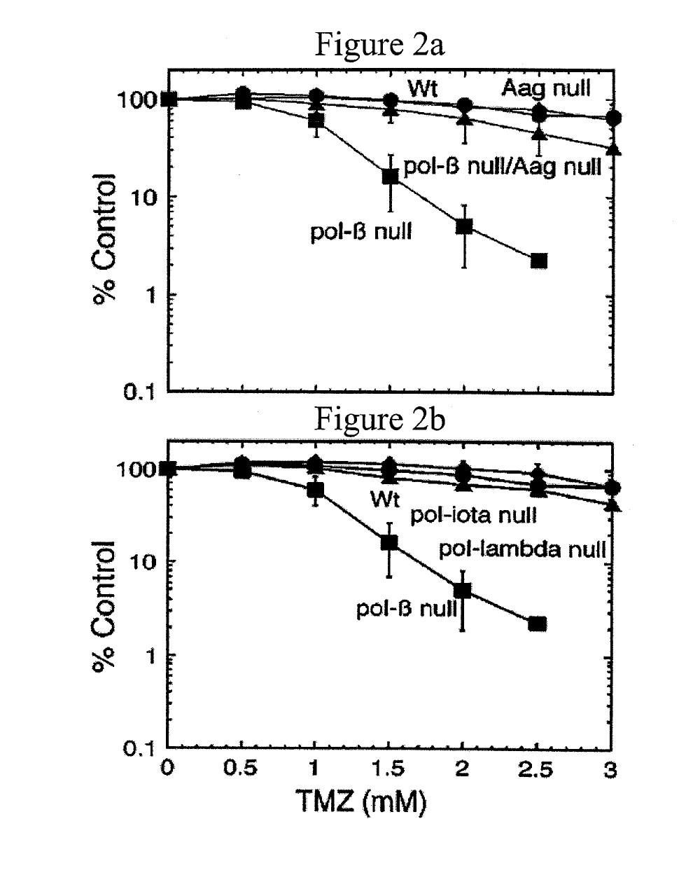

Enhanced TMZ-induced cytotoxicity in cells deficient in pol-β. To determine whether methylated base lesions induced by TMZ are repaired by the pol-β-dependent BER pathway, the cytotoxicity of TMZ in isogenic Wt, pol-β null, Aag null and pol-β/Aag double null cell lines was compared. TMZ exposure conferred no increase in cytotoxicity in Aag null MEF cells (FIG. 2a). However, pol-β null cells were significantly more sensitive to TMZ than either Wt or Aag null cells (FIG. 2a). Interestingly, pol-β/Aag double null cells were also resistant to TMZ, suggesting that the TMZ-induced hypersensitivity of pol-β deficient cells is dependent upon glycosylase-mediated initiation of repair (FIG. 2a) and that pol-β protects against accumulation of toxic BER intermediates (e.g., 5′dRP lesions).

Although pol-β is the predominant polymerase participating in BER both in vitro and in vivo, both pol-λ and pol-ι encode a nucleotidyl transferase activity and a 5′dRP lyase activity, similar to that found for pol-β, suggesting these polymerases may participate in BER. Therefore, cell lines deficient in the expression of pol-β, pol-λ, or pol-ι were directly compared to define their role in BER in vivo. Only pol-β null cells are hypersensitive to TMZ whereas cells deficient in either pol-λ, or pol-ι presented a Wt phenotype (FIG. 2b). Similar results were obtained for the alkylating agent MMS. As expected, these cell lines were equally sensitive to the cross-linking agent MMC.

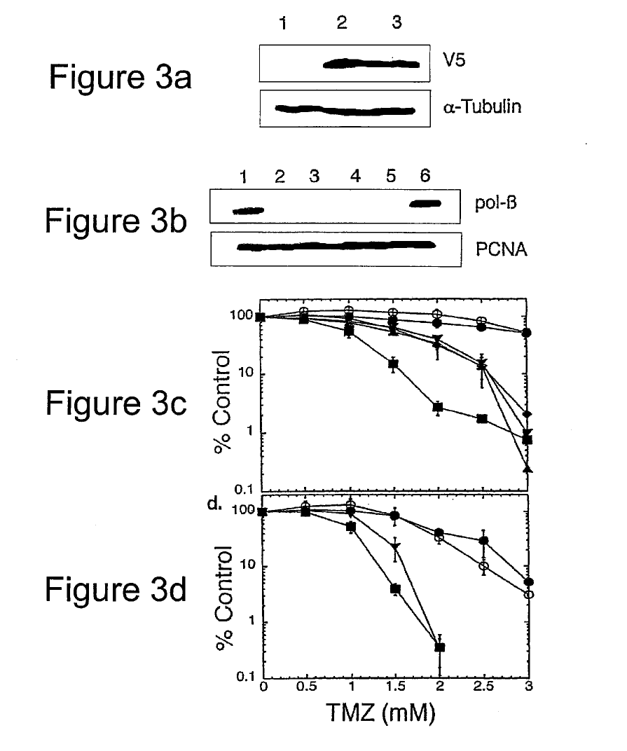

siRNA mediates long-term pol-β knockdown and induces a TMZ hypersensitive phenotype. The above findings suggest that inhibiting pol-β expression (or activity) would be sufficient to cause an accumulation of cytotoxic BER intermediates and thereby improve TMZ efficacy. RNAi-mediated gene knockdown was sufficient to effect a pol-β null phenotype (e.g., TMZ hypersensitivity). To evaluate siRNA-expressing plasmids for the ability to cause pol-β down-regulation, 293T cells were transiently transfected with both a V5-pol-β expression vector and pol-p-specific siRNA-expressing plasmids. The effect of siRNA on the expression of transfected pol-β is shown in FIG. 3a: pSuper.mpol-β787/805 caused strong inhibition of expression of the transfected V5-pol-β. Next, whether pSuper.mpol-β787/805 could lead to significant knockdown of endogenous pol-β was determined. Wt cells expressing pSuper.mpol-β787/805 lost from 80 to 99.9% pol-β protein expression (FIG. 3b), were measured by densitometric scanning.

The cytotoxic effect of TMZ in Wt and pol-β null cells was compared with three independently isolated clones (clones 2, 3 and 4) of Wt cells expressing pSuper.mpol-β787/805 and a Wt cell harboring a control pSuper plasmid. The siRNA-mediated knockdown of pol-β protein expression was sufficient to increase sensitivity to TMZ whereas control cell lines presented a Wt cell phenotype (FIG. 3c). However, the increase in sensitivity was modest. Increasing TMZ exposure from 2 hr to 48 hr increased formation of the active TMZ methylating species and increased formation of cytotoxic BER intermediates in the pol-β knockdown cells. With this longer treatment time, TMZ-induced cytotoxicity for the knockdown clones was similar to pol-β null cells (FIG. 3d). Similar results were obtained for the alkylating agent MMS and all cell lines were equally sensitive to MMC. Thus, it is concluded that siRNA is effective as a long-term down-regulator of pol-β and that this down-regulation leads to an increased sensitivity to TMZ.

Aag over-expression increases the TMZ hypersensitive phenotype. A second approach to increase the formation of toxic BER intermediates is to increase BER initiation via over-expression of human Aag. Increasing Aag expression promotes an increase in induced cytotoxicity in the absence of pol-β. Two Wt^{hAag} cell clones (Wt cells over-expressing hAag) and two Wt^{hAag/polβ-KD.4} cell clones (Wt pol-β knockdown cells over-expressing hAag) were isolated for further study; all four clones expressed equivalent levels of human Aag protein as determined by immunoblot analysis. The parental Wt, pol-β null, Wt^{Cont} and Wt^{Polβ-KD.4} cells expressed low levels of Aag activity; as measured by a standard in vitro glycosylase assay. However, the hAag over-expressing cells harbor a 20- to 35-fold increase in Aag activity (measured at 30 minutes incubation time) as compared to the parental cells.

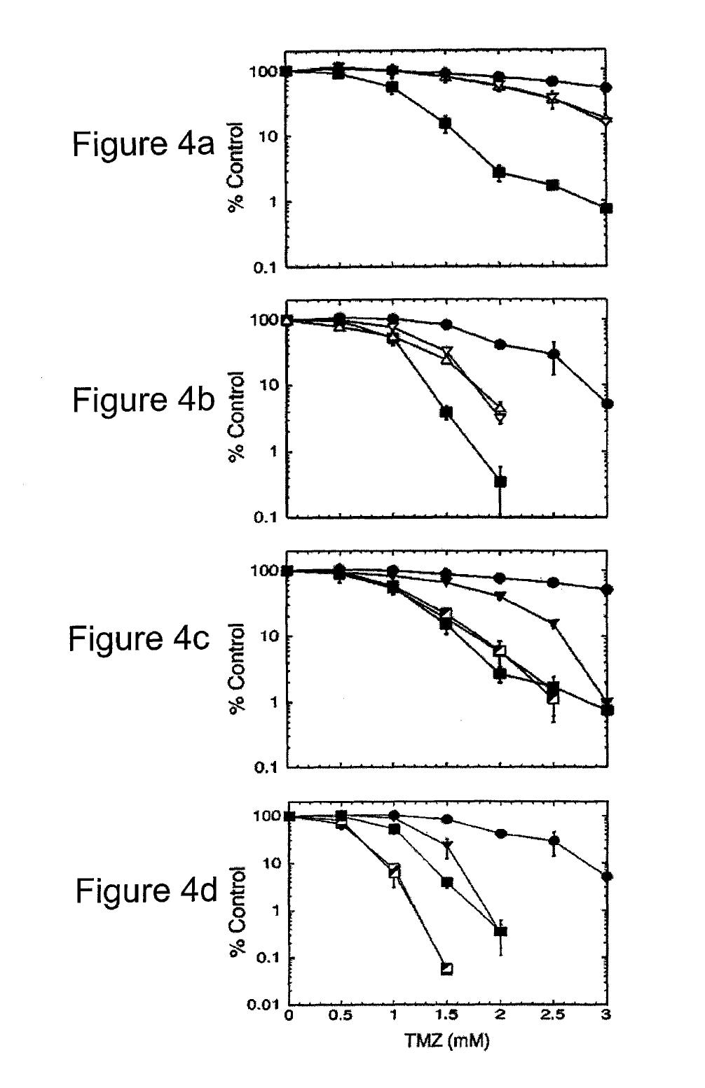

The elevated level of hAag appears to have generated increased levels of cytotoxic BER intermediates following TMZ exposure, as both wt^{hAag3} and Wt^{hAag8} cells presented a slight TMZ hypersensitivity with 2 hr exposure and an increased hypersensitivity with 48 hr exposure (FIGS. 4a,b). The most prominent effect was observed upon the combination of pol-β knockdown and hAag over-expression (wt^{hAag.2/polβ-KD.4} and wt^{hAag.3/polβ-KD.4} cells, FIGS. 4c,d). These cells exhibited a pol-β null-like phenotype when exposed to TMZ for 2 hr, a significant increase in TMZ efficacy as compared to the parental Wt^{Polβ-KD.4} cells, presenting an IC₅₀ of 1.25 mM, similar to pol-β cells (FIG. 4c). Further, when exposed to TMZ for 48 hr, TMZ efficacy was improved almost 4-fold (FIG. 4d); the IC₅₀ for Wt cells is 2.6 mM whereas the IC₅₀ for wt^{hAag.3/Polβ-KD.4} cells is approximately 0.7 mM. Similar results were obtained for MMS. As with the other cell lines, these cells were all equally sensitive to MMC.

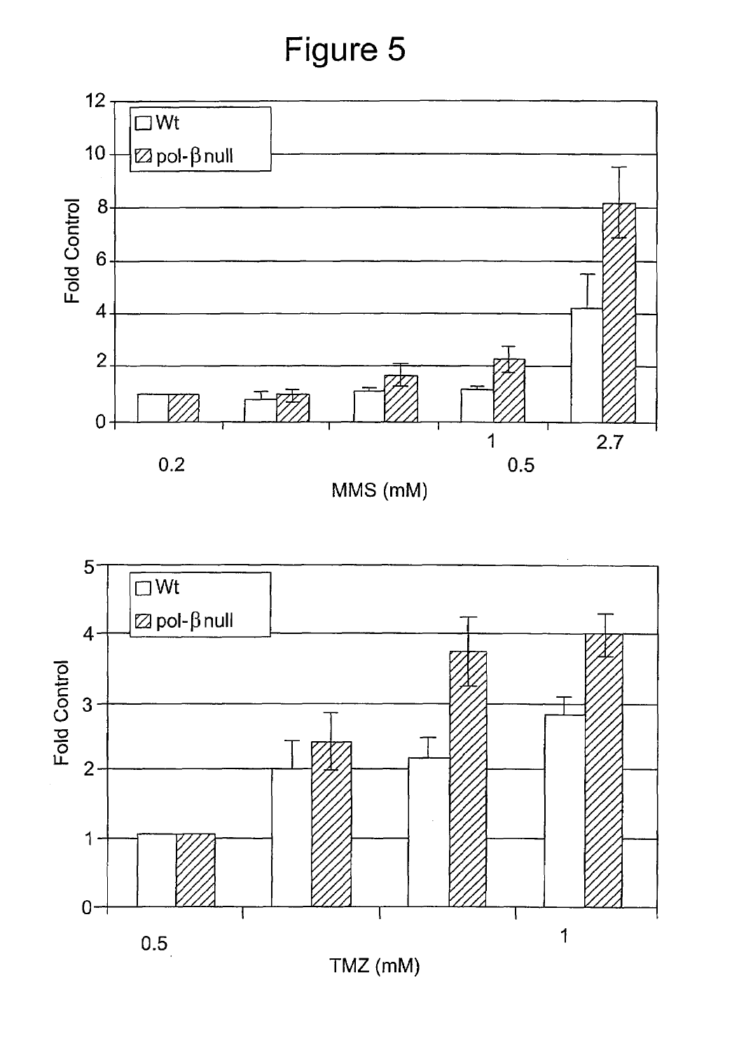

Increased phosphorylation of γ-H2AX in pol-β deficient cells exposed to TMZ. Pol-β is essential for BER in vivo and failure to repair alkylation-damage induced BER intermediates leads to cell death. However, the mechanism of BER intermediate induced cell death has not been determined. It was therefore investigated whether accumulation of BER intermediates (e.g., 5′dRP) leads to activation of the DNA damage/replication checkpoint response by monitoring the expression of γ-H2AX following both MMS and TMZ treatment in Wt and pol-β null cells. The results indicate that that pol-βdeficient cells exhibited a clear increase in γ-H2AX expression as compared to Wt cells following MMS and TMZ (FIG. 5) exposure. Quantitative analysis of MMS and TMZ induced H2AX phosphorylation is shown in FIG. 5. The immunoblots demonstrate MMS and TMZ induced phosphorylation of γ-H2AX.

### Example 2

This example demonstrates that inhibition of DNA polymerase beta increases sensitivity to TMZ in human cells in vitro.

### Materials and Methods

Chemicals and Reagents. RPMI 1640 and heat inactivated fetal bovine serum were from Cambrex Biosciences Group, (Walkersville, Md.) and InVitrogen-Gibco (Carlsbad, Calif.). TMZ was from the National Cancer Institute Developmental Therapeutics Program and prepared as a 100 mM stock in DMSO. The following primary antibodies were used: anti-pol-β (Mab clone 61; NeoMarker, Fremont, Calif.); anti-human Mpg (Mab; clone 506-3D) was kindly provided by Dr. S. J. Kennel (ORNL); anti-Ape1 (EMD Biosciences, Inc, San Diego, Calif.) anti-proliferating cell nuclear antigen (PCNA; Santa Cruz Biotechnology, Santa Cruz, Calif.); and anti-Flag (M2 Mab; Sigma-Aldrich, Saint Louis, Mo.). All electrophoresis reagents were from Bio-Rad (Hercules, Calif.). Neomycin and Dynabeads Protein G were purchased from Invitrogen-Gibco (Carlsbad, Calif.). Puromycin, Gentamicin sulfate solution (10 mg/ml) and 3× Flag peptide were from BD Clontech (Mountain View, Calif.), Irvine Scientific (Santa Ana, Calif.) and Sigma-Aldrich (Saint Louis, Mo.) respectively.

Plasmid Expression Vectors and RNAi Development. The following mammalian expression vectors were used: human Mpg: pRS1422; human pol-β: pIRES-Neo/Flag-pol-β (Wt), pIRES-Neo/Flag-pol-β (D256A) and pIRES-Puro/Flag-pol-β (Wt); each were described previously (Sobol et al., J. Biol Chem; 278(41):39951-59 (2003) and Sobol et al., Nature; 405(6788), 807-10 (2000)). Human pol-β targeted shRNA expression vectors (FIV-based lentiviral vectors) were designed using an RNAi design algorithm from System Biosciences (Mountain View, Calif.).

Cell Lines Transfection and Viral Transduction Conditions. Human Mpg, human Flag pol-β and human Flag pol-β (D256A) over-expressing cell lines were prepared as follows: briefly, 1.5×10⁵ cells were seeded into 60 mm dishes and incubated for 24-30 hours at 5% CO2 at 37° C. The human Mpg expression plasmid (pRS1422) and human pol-β expression plasmids [pIRES-Neo/Flag-pol-β (Wt), pIRES-Neo/Flag-pol-β (D256A) and pIRES-Puro/Flag-pol-β (Wt)] were transfected using FuGene 6 Transfection Reagent (Roche Diagnostic Corp, Indianapolis, Ind.) according to the manufacturer's instructions. Stable cell lines were selected in G418 (800 μg/ml for human Mpg expression plasmids, 700 μg/ml for pIRES-Neo/Flag-pol-β (Wt) and pIRES-Neo/Flag-pol-β (D256A) and puromycin (0.5 μg/ml) for pIRES-Puro/Flag-pol-β (Wt) for 2 weeks. Individual clones (stably expressing human Mpg or pol-β protein) were amplified and 30 μg of nuclear extract was analyzed by immunoblotting for the expression of human Mpg or human pol-β protein using the Nucbuster nuclear protein extraction reagent (EMD Biosciences, Inc, San Diego, Calif.) and also probed for expression of Ape1 and PCNA.

Infectious lentiviral particles were generated as follows: briefly, 12×10⁶ cells (293-FT cells, InVitrogen) were seeded into each of three separate 150 mm dishes and incubated for 24-30 hours at 5% CO₂ at 37° C. Twenty μg of the shRNA expression plasmid [pFIV-H1(hpolB1)-puro, pFIV-H1(hpolB2)-puro, pFIV-H1(hpolB3)-puro or pFIV-H1(hpolB1)-copGFP] was then co-transfected into the 293-FT cells together with 20 μg of each of the packaging vectors pVSV-G and pFIV-34N (Peschla et al., Nat Med; 4(3):354-57 (1998)) using FuGene 6 Transfection Reagent (Roche Diagnostic Corp, Indianapolis, Ind.) according to the manufacturer's instructions. After 48 h the viral supernatant from each dish was harvested, pooled and filtered (0.45 μM). One ml aliquots of virus stock were stored at −80° C. until use.

Viral transduction was completed as follows: Briefly, 6.0×10⁴ cells were seeded into 6-well plate and incubated for 24-30 hours at 5% CO₂ at 37° C. Cells were transduced for 18 hours with shRNA-expressing lentiviral stocks at 32° C., media was changed and the cells were further cultured for 72 h at 37° C. Stable cell lines were isolated following transduction as above followed by selection in puromycin (0.5 μg/ml) for 2 weeks. Individual clones were amplified and 30 μg of nuclear extract was analyzed by immunoblotting to monitor the expression of endogenous human pol-β protein and related nuclear proteins (Mpg, Ape1 and PCNA) using the Nucbuster nuclear protein extraction reagent (EMD Biosciences, Inc, San Diego, Calif.).

Culture Conditions and Cell Cytotoxicity Assays. Human Mpg over-expression, human pol-β knockdown and human pol-β over-expressing MDA-MB-231 breast cancer cell lines were cultured at 37° C. in a humidified incubator with 5% CO₂ in RPMI 1640 (Cambrex Biosciences Group, Walkersville, Md.) supplemented with 10% heat inactivated fetal bovine serum and gentamicin (10 μg/ml). Temozolomide induced cytotoxicity was determined by growth inhibition assays. Briefly, cells were seeded in 96-well dishes at 2000 cells/well. After 24 hours, cells (quadruplicate wells) were exposed to serial dilutions of Temozolomide in growth media at 37° C. for 48 hours at which point the total cell number was determined by a modified 3-(4,5-dimethylthiazol-2-yl)-2,5-diphenyltetrazolium bromide (MTT) assay (MTS; Promega Madison, Wis.) (Cory et al., Cancer Communications; 3(7):207-12 (1991)). Metabolically active cells were quantified by the bioreduction of the MTS tetrazolium compound by recording absorbance at 490 nm using a microplate reader. Results were calculated from the average of four separate experiments and are reported as the % of treated cells relative to the cells in control wells (% Control).

Cell Extract Preparation, Immunoblot and Immunoprecipitation Assays. Nuclear extracts were prepared using the NucBuster nuclear protein extraction reagent (EMD Biosciences, Inc, San Diego, Calif.). Protein concentration was determined using Bio-Rad protein assay reagents according to the manufacturer's instruction. Nuclear protein (30 μg) was separated by electrophoresis in a 4-20% Tris-Glycine SDS-polyacrylamide gel (Invitrogen; Carlsbad, Calif.) and electro-transferred to a 0.45 μM nitrocellulose membrane (Trans-Blot, Bio-Rad; Hercules, Calif.). Antigens were detected using standard protocols. Primary antibodies (anti-pol-β, 500×; anti-hMpg, 1000×; anti-Ape1, 3000×; anti-Flag 2000× and anti-PCNA, 1000×) and the horseradish peroxidase (HRP)-conjugated secondary antibody (goat anti-mouse HRP; Bio-Rad; Hercules, Calif.) were diluted in TBST/5% milk.

For immunoprecipitation (IP), cell lysate from the above cell lines was prepared in RIPA buffer incubated overnight with anti-Flag antibodies (Sigma-Aldrich) at 4° C. followed by 1-h incubation with Protein G Dynabeads at 4° C. Immune complex was washed with RIPA buffer and immunoprecipitates were eluted with 3× Flag peptide (Sigma-Aldrich), separated on SDS-PAGE and transferred to nitrocellulose filters and probed with anti-pol-β antibody.

### Results

Having demonstrated that RNA interference specific to mouse pol-β can lead to chemotherapeutic agent hypersensitivity in mouse cells (Example 1 and Trivedi et al., Cancer Res; 65(14):6394-400 (2005)), the role of pol-β in TMZ-responsiveness in the human breast cancer cell line MDA-MB-231 (a p53-cell line) was next evaluated. As in mouse fibroblasts, de-regulation of the BER pathway in human tumor cells results in significant increases in TMZ-responsiveness in cells in culture and that the increase in TMZ-mediated hypersensitivity is due to un-repaired 5′dRP lesions, the specific substrate of pol-β (Sobol et al., Nature, 405:807-810, 2000).

RNAi-mediated knockdown of human pol-β: To facilitate the analysis of the BER pathway and pol-β in particular, in human tumor cells, 5 separate shRNA expressing vectors (plasmid and lentiviral-based) were designed which were specific for different regions of pol-β mRNA, designed using either the Oligoengine or Systems Biosciences shRNA design algorithm. As shown in FIG. 6d, four sequences predicted to be effective shRNA targets are within the open reading frame (ORF) and one sequence was specific for a region outside the ORF, within the 3′UTR. Each plasmid or lentiviral vector was used to develop 20 separate single-cell clones and each was analyzed for effectiveness of pol-β knockdown by probing pol-β expression by immunoblot analysis of nuclear proteins. Interestingly, the ORF specific shRNA vectors were only partially or minimally effective in leading to a decrease in pol-β expression. However, all 20 cell clones expressing the 3′UTR specific pol-β shRNA led to complete loss of pol-β protein expression (FIG. 7). Note that the expression of the human pol-β specific shRNA does not effect the expression of the other BER proteins Mpg or Ape1 (FIG. 7). PCNA expression is shown as a loading control.

Increased temozolomide sensitivity in Human Breast Cancer cells after pol-β knockdown mediated by expression of pol-β specific siRNA/shRNA: Five siRNA/shRNA expression plasmids, specific to human pol-β (based on Genbank seq. NM_{—}002690) were designed using several different siRNA/shRNA algorithms. Of those, 4 were specific for the human pol-β ORF and one was specific for the human pol-β 3′UTR. Interestingly, the 3′UTR targeted siRNA proved most effective. The sequence for this is shown in FIG. 6a. This shRNA is expressed via a robust lentiviral delivery system.

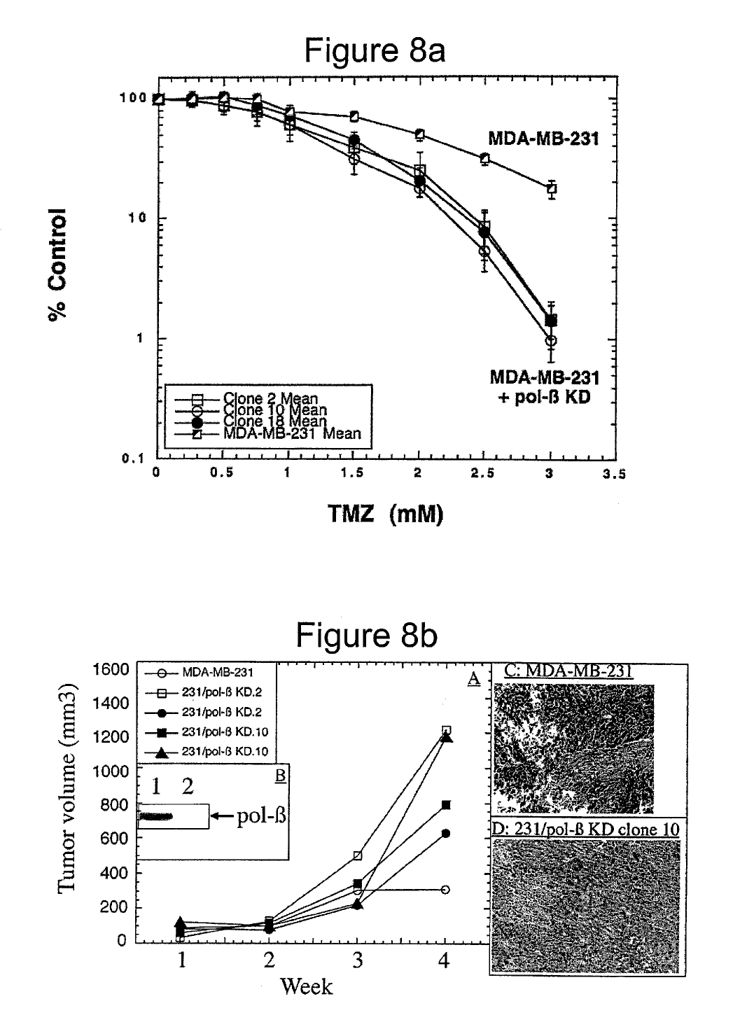

Lentiviral expressed shRNA mediates long-term and stable knockdown of pol-β: Loss of expression has been observed for cells in culture as long as 4 months, which have not been analyzed after longer periods in culture. The cells were next tested for TMZ sensitivity so as to evaluate the impact of the loss of pol-β. Human tumor cells that have lost pol-β expression were significantly more sensitive to TMZ than the parental cells (FIG. 8a). These results also demonstrate, for the first time, that TMZ-hypersensitivity due to pol-β deficiency in human cells is independent of p53.

Many of the cell lines were chosen because of their clinically-representative genetic variability and because many can be grown as tumor xenografts in nude mice. Although loss of pol-β has no effect on the rate of cell growth when cells are grown in culture, the MDA-MB-231 parental cells and the derived pol-β knockdown cells were implanted into the right flank of nude mice to determine if loss of pol-β alone effected tumor growth. However, as shown, loss of pol-β had no impact on tumor growth as human tumor xenografts (FIG. 8b). Most importantly, the shRNA-mediated deficiency in pol-β in these tumor cells was maintained while the cells were grown as a tumor. Analysis of the isolated tumor xenograft showed little or no detectable pol-β, as determined by immunoblot analysis of nuclear proteins extracted from the tumor (FIG. 8b, panel B). In addition, expression of pol-β by immunohistochemical analysis of the extracted WT MDA-MB-231 derived tumor (FIG. 8b, panel C) was observed, but tumors derived from the shRNA-mediated pol-β knockdown cells (231/pol-β KD) presented with reduced or absent pol-β expression (FIG. 8b, panel D).

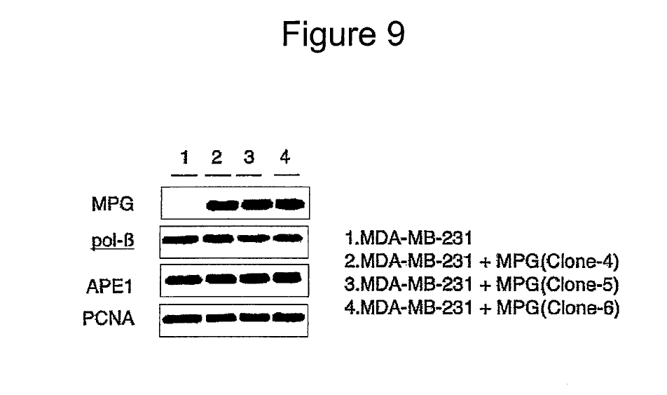

Regulating Aag/Mpg expression in human tumor cells alters TMZ responsiveness: The Aag/Mpg expression system described herein affords the ability to significantly over-express WT Aag/Mpg, as well as point mutants of Aag/Mpg in human cells (FIG. 9), with activity due to over-expression of wt AAG increasing as much as 35-fold Trivedi et al., Cancer Res; 65(14):6394-400, 2005. Note almost no detectable expression of Aag/Mpg in the parental cells as compared to the over-expression cell lines (Compare lane 1 with lanes 2-4; FIG. 9) and that the over-expression of Aag/Mpg does not effect the expression of the other BER proteins pol-β or Ape1 (FIG. 9). PCNA expression is shown as a loading control.

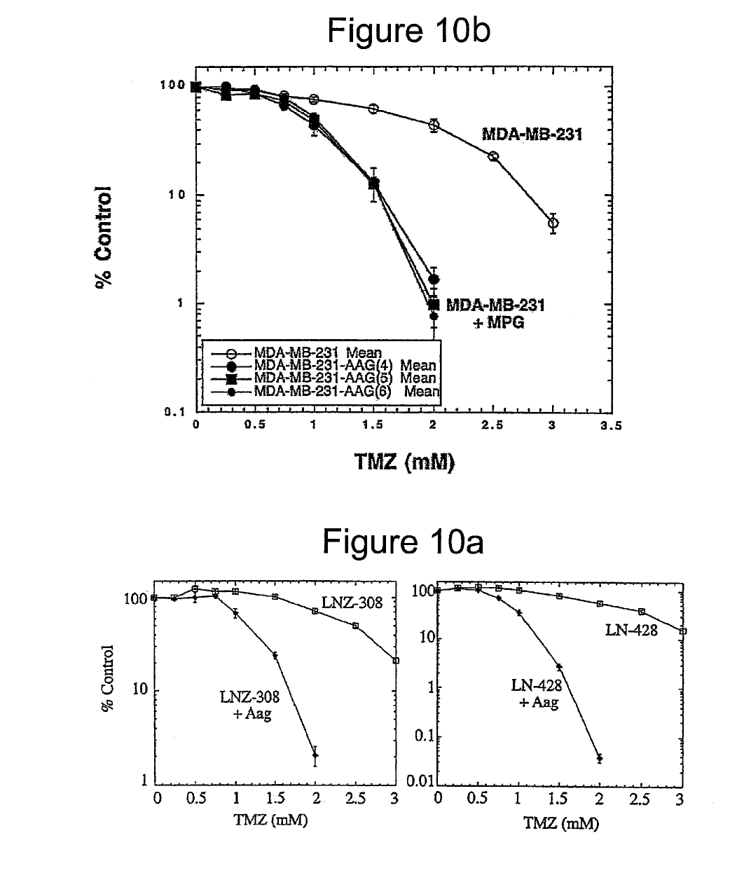

Over-expression of Aag/Mpg shifts the rate-limiting step in the BER pathway, significantly enhancing BER initiation. Without wishing to be bound by any particular theory, it is believed that Aag/Mpg over-expression then promotes the accumulation of the BER intermediate 5′dRP. This accumulation of un-repaired 5′dRP then leads to the increase in cell death following exposure to TMZ. This increased sensitivity to TMZ when Aag/Mpg is over-expressed is observed in LNZ-308 glioblastoma cells, harboring a deletion in the TP53 and PTEN genes (FIG. 10a, left panel); in LN-428 glioblastoma cells, harboring mutations in TP53 but a wt PTEN gene (FIG. 10a, right panel) and in the p53 deficient breast cancer cell line MDA-MB-231 (FIG. 10b), indicating that the increase in TMZ-responsiveness due to an alteration in BER capacity (e.g., Aag/Mpg over-expression) is not cell type specific and may well be independent of both p53 and PTEN. It should be noted here that Rinne et al have also demonstrated that Aag/Mpg over-expression increased the sensitivity of breast cancer cells to TMZ (Rinne et al., Mol Cancer Ther; 3(8):955-67 (2004)).

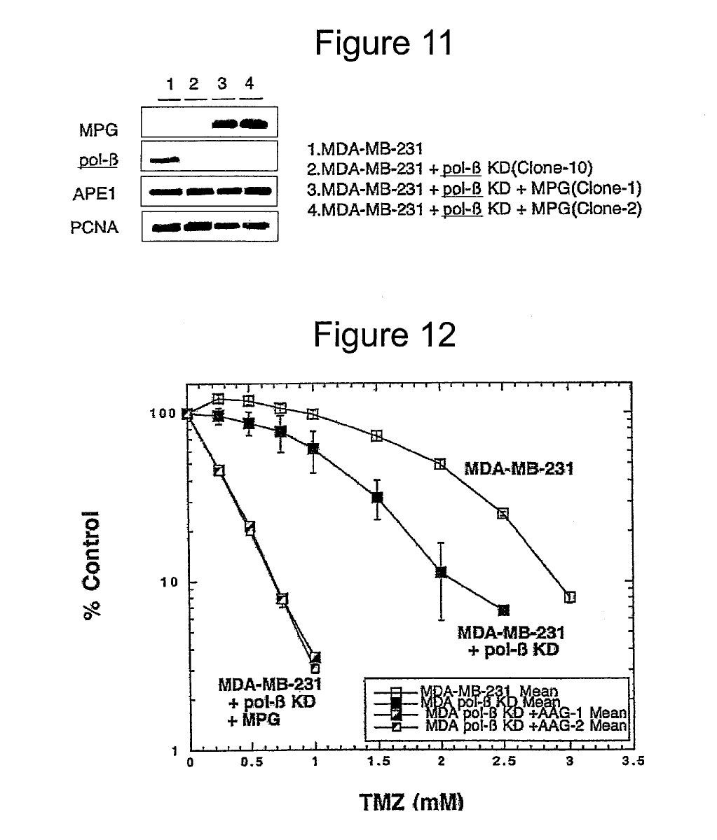

Combined Mpg/Aag over expression and shRNA-mediated pol-β knockdown: Since Mpg/Aag performs the first step in the BER pathway when repairing alkylated bases and is essential to manifest the pol-β null phenotype in mouse cells (Sobol et al., J. Biol Chem; 278(41):39951-59 (2003)), Mpg/Aag over-expression and pol-β knockdown was combined. Immunoblots are shown demonstrating that these human tumor cells can harbor both Mpg/Aag over-expression plus the loss of pol-β expression (FIG. 11). Functionally, over-expression of Mpg/Aag combined with pol-β knockdown drastically increased the sensitivity of human tumor cells to TMZ, a decrease in IC₅₀ of almost 10-fold when compared to the parental cells, as shown (FIG. 12). The increase in TMZ sensitivity due to pol-β knockdown and the further increase in TMZ sensitivity when pol-β knockdown is combined with Aag/Mpg over-expression underscores the important role pol-β and BER plays in protection from TMZ induced cell death.

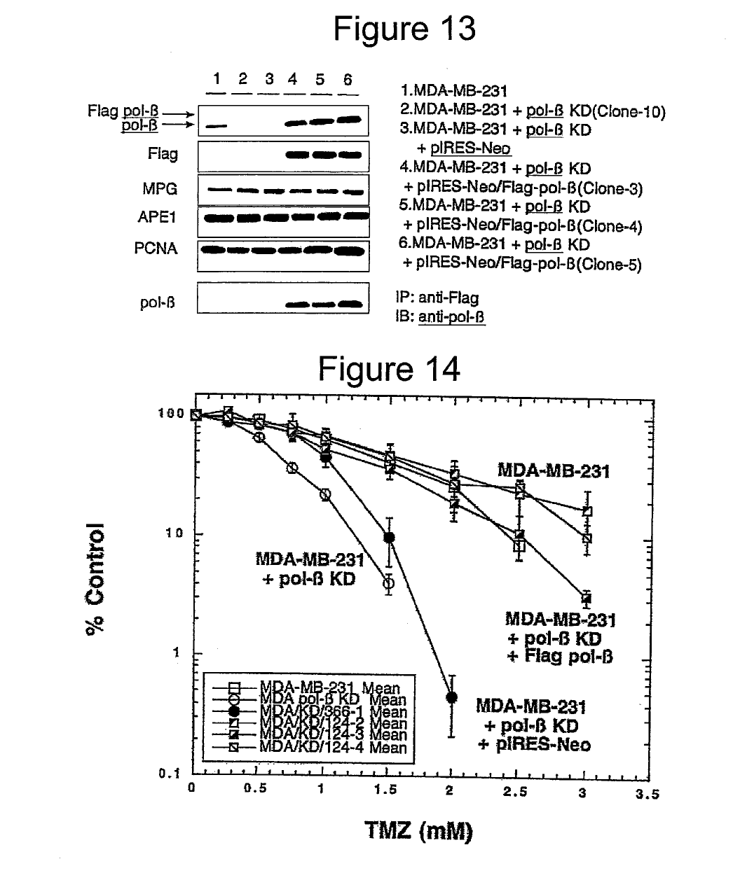

Hypersensitivity of human pol-β knockdown cells is due to un-repaired 5′dRP lesions: The results of these experiments reveal that loss of pol-β expression, as mediated by shRNA expression, leads to hypersensitivity to TMZ (FIG. 8). To demonstrate that this observation is due to specific loss of pol-β and not due to an aberrant RNAi-mediated non-specific effect, the pol-β knockdown cells were complemented with human epitope-tagged pol-β (FIG. 13). This is feasible by taking advantage of the 3′UTR-specific pol-β shRNA system (FIG. 6a) and to compliment pol-β knockdown human tumor cells using expression vectors lacking the shRNA-targeted 3′UTR. As shown, there is no expression of endogenous pol-β but robust expression of the transgenic Flag-pol-β (FIG. 13). Also, note that there is no change in the expression of the other BER proteins Mpg or Ape1 (FIG. 13). PCNA expression is shown as a loading control. Importantly, expression of wild-type Flag-pol-β in the human tumor pol-β knockdown cells completely restores resistance to TMZ (FIG. 14) whereas cells with the vector control are just as sensitive to TMZ as the original pol-β knockdown cells.

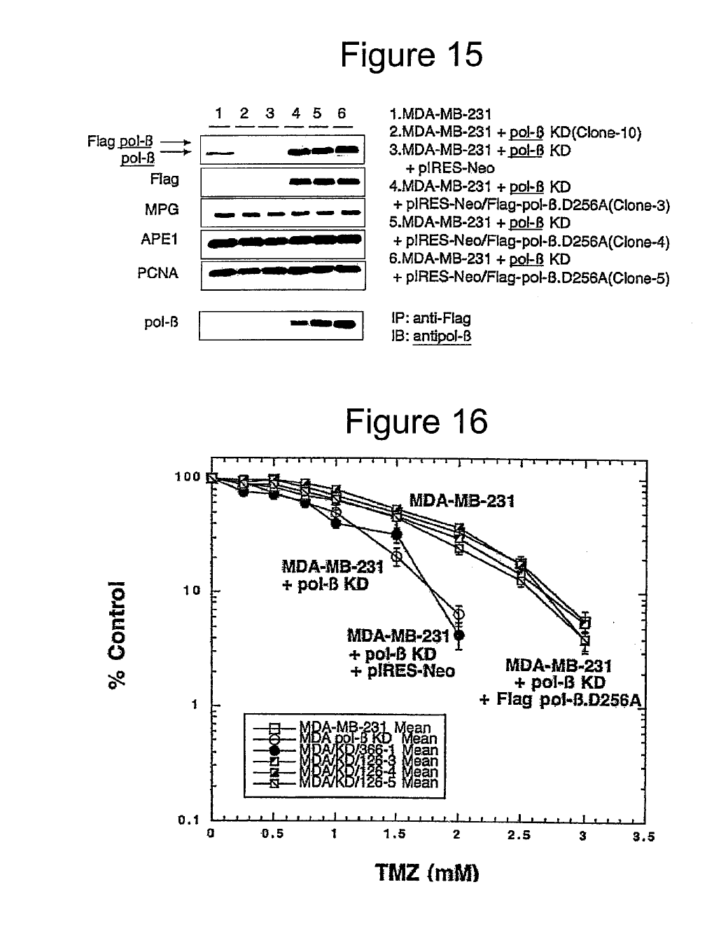

However, as has been shown previously, the pol-β substrate and BER intermediate 5′dRP is highly toxic if left un-repaired (Sobol et al., Nature; 405(6788):807-10 (2000); Sobol et al., Journal of Biological Chemistry; 278: 39951-39959 (2003); Sobol et al., Nature, 379:183-186, (1996)), suggesting that the observed hypersensitivity of human pol-β knockdown cells (FIG. 8) to TMZ may be due to loss of the 5′dRP lyase activity of pol-β. To this end, the pol-β knockdown cells was complemented with human epitope-tagged pol-β with a D256A mutation in the polymerase active site that retains complete 5′dRP lyase activity (FIG. 15). As with the cells complemented with wt pol-β, there was no expression of endogenous pol-β but robust expression of the transgenic Flag-pol-β D256A mutant (FIG. 15). Also, there was no change in the expression of the other BER proteins Mpg or Ape1 (FIG. 15). PCNA expression is shown as a loading control. Most importantly, expression of the polymerase-defective/5′dRP lyase active D256A pol-β mutant in the human tumor pol-β knockdown cells completely restored resistance to TMZ (FIG. 16) whereas cells with the vector control were just as sensitive to TMZ as the original pol-β knockdown cells, indicating that the hypersensitivity of cells to TMZ that have lost pol-β expression (due to pol-β specific shRNA) is due to un-repaired cytotoxic 5′dRP lesions.

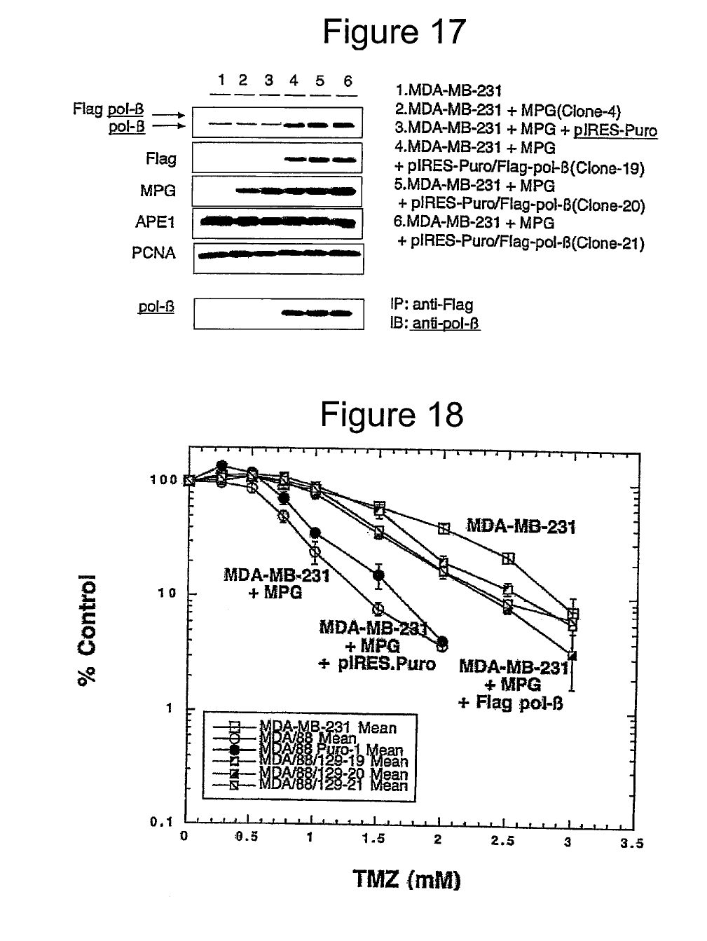

Hypersensitivity of cells over-expressing Mpg/Aag is reversed by pol-β expression: The mechanism behind the observed hypersensitivity of human tumor cells to TMZ when Mpg is over-expressed (FIG. 10) is unknown. However, since Mpg over-expression increases the hypersensitivity of pol-β knockdown cells (FIG. 12), it is possible that Mpg over-expression saturates endogenous pol-β and yields an apparent pol-β deficiency, since Mpg over-expression could cause an overall BER imbalance. To determine if the endogenous level of pol-β is rate limiting when Mpg is over-expressed, the Mpg over-expression MDA-MB-231 human breast cancer cells were modified to also present with over-expression of pol-β. For this, the Mpg over-expression cells were complemented with human epitope-tagged pol-β (FIG. 17). There was no change in expression of endogenous pol-β but robust expression of the transgenic Flag-pol-β (FIG. 17). Also, there was no change in the expression of the other BER proteins (FIG. 17). PCNA expression is shown as a loading control. However, expression of wild-type pol-β in the human tumor Mpg over-expression cells completely restores resistance to TMZ (FIG. 18), whereas cells with the vector control were just as sensitive to TMZ as the original cells, demonstrating that the hypersensitivity of cells to TMZ that over-express Mpg is due to un-repaired pol-β substrates such as the cytotoxic 5′dRP lesions that would accumulate following TMZ exposure.

Summary: Overall, these results demonstrate that pol-β and BER contributes significantly to the repair of TMZ-induced DNA damage and that modulating the BER pathway by pol-β inhibition or loss of expression enhances the chemotherapeutic index of agents, such as TMZ, that damage DNA and initiate BER.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. For example, by “first, second, third, fourth, fifth, tenth, twentieth, fiftieth, hundredth five-hundredth, thousandth, etc.” it is specifically meant that integer values not recited between “first” and “thousandth” are included, and by “etc.” it is intended that values over “thousandth” are contemplated, such as two thousandth, five thousandth, ten thousandth, and so on, as are intervening integer values (e.g., three thousandth, two hundred twenty-fifth).

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein, including in the following bibliography, which are not hereby admitted to be prior art (some of which have published since the priority date), are hereby incorporated by reference to the same extent as if each reference were individually and specifically indicated to be incorporated by reference and were set forth in its entirety herein.

### BIBLIOGRAPHY

Albertella et al., “The overexpression of specialized DNA polymerases in cancer,” DNA Repair, 4, 583-93, 2005.
Bakkenist et al., “DNA damage activates ATM through intermolecular autophosphorylation and dimer dissociation,” Nature, 421: 499-506, 2003.
Barvaux et al., “Dual repair modulation reverses Temozolomide resistance in vitro,” Molecular Cancer Therapeutics, 3:123-127, 2004.
Bebenek et al., “5′-Deoxyribose phosphate lyase activity of human DNA polymerase iota in vitro” Science, 291: 2156-2159, 2001.
Behin e al., “Primary brain tumours in adults,” Lancet, 361-323-331, 2003.
Berridge and Tan, “Characterization of the cellular reduction of 3-(4,5-dimethylthiazol-2-yl)-2,5-diphenyltetrazolium bromide (MTT): subcellular localization, substrate dependence, and involvement of mitochondrial electron transport in MTT reduction,” Archives of Biochemistry and Biophysics, 303:474-482, 1993.
Bobola et al., “Apurinic endonuclease activity in adult gliomas and time to tumor progression after alkylating agent-based chemotherapy and after radiotherapy,” Clinical Cancer Research; 10(23):7875-83, 2004.
Bobola et al., “Contribution of 06-methylguanine-DNA methyltransferase to resistance to 1,3-(2-chloroethyl)-1-nitrosourea in human brain tumor-derived cell lines,” Molecular Carcinogenesis, 13:81-88, 1995.
Bobola et al., “Contribution of 06-methylguanine-DNA methyltransferase to monofunctional alkylating-agent resistance in human brain tumor-derived cell lines,” Molecular Carcinogenesis, 13: 70-80, 1995.
Bobola et al., “Role of 06-methylguanine-DNA methyltransferase in resistance of human brain tumor cell lines to the clinically relevant methylating agents temozolomide and streptozotocin,” Clinical Cancer Research, 2:735-741, 1996.
Bocangel et al., “Multifaceted resistance of gliomas to temozolomide,” Clinical Cancer Research, 8:2725-2734, 2002.
Brown and Baltimore, “Essential and dispensable roles of ATR in cell cycle arrest and genome maintenance,” Genes & Development, 77:615-628, 2003.
Chahlavi et al., “Impact of chromosome 1 p status in response of oligodendroglioma to temozolomide: preliminary results, Journal of Neurooncology, 61:267-273, 2003.
Chaturvedula et al., “A New Acylated Oleanane Triterpenoid from Couepia polyandra that Inhibits the Lyase Activity of DNA Polymerase,” J. Nat. Prod., 66, 1463-1465, 2003.
Chaturvedula et al., “A new ursane triterpene from Monochaetum vulcanicum that inhibits DNA polymerase beta lyase,” J. Nat. Prod., 67(5), 899-901, 2004.
Chaturvedula et al., “New lupane triterpenoids from Solidago canadensis that inhibit the lyase activity of DNA polymerase beta,” Bioorg Med Chem., 12(23), 6271-75, 2004.
Chaturvedula et al., “New neolignans that inhibit DNA polymerase beta lyase,” J. Nat. Prod., 67(6), 964-67, 2004.
Chen et al., “Response to RAG-mediated VDJ cleavage by NBS1 and gamma-H2AX,” Science, 290:1962-65, 2000.
Christodoulou et al., “Temozolomide (TMZ) combined with cisplatin (CDDP) in patients with brain metastases from solid tumors: a Hellenic Cooperative Oncology Group (HeCOG) Phase II study,” Journal of Neurooncology, 71:61-65, 2005.
Cory et al., “Use of an aqueous soluble tetrazolium/formazan assay for cell growth assays in culture,” Cancer Communications, 3(7):207-12 (1991).
Deng et al., “A new 7,8-euphadien-type triterpenoid from Brackenridgea nitida and Bleasdalea bleasdalei that inhibits DNA polymerase beta,” J. Nat. Prod.; 63(10):1356-60, 2000.
Deng et al., “DNA polymerase beta inhibitors from Baeckea gunniana,” J. Nat. Prod., 62(12), 1624-26, 1999.
Dinnes et al., “A rapid and systematic review of the effectiveness of temozolomide for the treatment of recurrent malignant glioma,” British Journal of Cancer, 86:501-505, 2002.
Ebert et al., “Use of temozolomide with other cytotoxic chemotherapy in the treatment of patients with recurrent brain metastases from lung cancer,” Oncologist, 8:69-75, 2003.
Eggermont and Kirkwood, “Re-evaluating the role of dacarbazine in metastatic melanoma: what have we learned in 30 years?” European Journal of Cancer, 40:1825-1836, 2004.
Engelward et al., “Repair-deficient 3-methyladenine DNA glycosylase homozygous mutant mouse cells have increased sensitivity to alkylation-induced chromosome damage and cell killing,” EMBO Journal, 15: 945-952, 1996.
Eton et al., “Prognostic factors for survival of patients treated systemically for disseminated melanoma,” Journal of Clinical Oncology, 16:1103-1111, 1998.
Friedman et al., “O6-benzylguanine-mediated enhancement of chemotherapy,” Molecular Cancer Therapeutics, 1:943-948, 2002.
Garcia-Diaz et al., “Identification of an intrinsic 5′-deoxyribose-5-phosphate lyase activity in human DNA polymerase lambda: a possible role in base excision repair,” Journal of Biological Chemistry, 276:34659-34663, 2001.
Gaya et al., The use of temozolomide in recurrent malignant gliomas,” Cancer Treatment Reviews, 28:115-120, 2002.
GenBank Accession No. NM_{—}002690, “Homo sapiens polymerase (DNA directed), beta (POLB), mRNA”
Grossman and Batara, “Current management of glioblastoma multiforme,” Seminars in Oncology, 31:635-644, 2004.
Haracska et al., “A mechanism for the exclusion of low-fidelity human Y-family DNA polymerases from base excision repair,” Genes & Development, 77:2777-2785, 2003.
Kokkinakis et al., “Thresholds of 06-alkylguanine-DNA alkyltransferase which confer significant resistance of human glial tumor xenografts to treatment with 1,3-bis(2-chloroethyl)-1-nitrosourea or temozolomide,” Clinical Cancer Research, 7:421-428, 2001.
Lindahl and Wood, “Quality control by DNA repair,” Science, 286:1897-1905, 1999.
   Liu and Gerson, “Therapeutic impact of methoxyamine: blocking repair of abasic sites in the base excision repair pathway,” Current Opinion in Investigative Drugs, 5:623-627, 2004.
Madhusudan et al., “The emerging role of DNA repair proteins as predictive, prognostic and therapeutic targets in cancer,” Cancer Treatment Reviews, 31, 603-617, 2005.
McDonald et al., “129-derived strains of mice are deficient in DNA polymerase iota and have normal immunoglobulin hypermutation,” Journal of Experimental Medicine, 198:635-643, 2003.
Miao et al., “3-Methyladenine-DNA glycosylase (MPG protein) interacts with human RAD23 proteins,” Journal of Biological Chemistry, 275:28433-28438, 2000.
Middleton et al., “Effect of 06-(4-bromophenyl) guanine on different temozolomide schedules in a human melanoma xenograft model,” International Journal of Cancer, 700:615-617, 2002.
Middleton et al., “Randomized phase III study of temozolomide versus dacarbazine in the treatment of patients with advanced metastatic malignant melanoma,” Journal of Clinical Oncology, 18: 158-166, 2000.
Paik et al., “Sensitization of human carcinoma cells to alkylating agents by small interfering RNA suppression of 3-alkyladenine-DNA glycosylase,” Cancer Res; 65(22):10472-7, 2005.
Paull et al., “A critical role for histone H2AX in recruitment of repair factors to nuclear foci after DNA damage,” Current Biology, 10: 886-895, 2000.
Poeschla et al., “Efficient transduction of nondividing human cells by feline immunodeficiency virus lentiviral vectors,” Nat Med, 4(3):354-7, 1998.
Polosina at al., “‘Knock down’ of DNA polymerase by RNA interference: recapitulation of null phenotype,” DNA Repair 3, 1469-74, 2004.
Prasad et al., “Localization of the deoxyribose phosphate lyase active site in human DNA polymerase iota by controlled proteolysis,” Journal of Biological Chemistry, 278:29649-29654, 2003.
Rappold et al., “Tumor suppressor p53 binding protein 1 (53BP1) is involved in DNA damage-signaling pathways,” Journal of Cell Biology, 753:613-620, 2001.
Rinne et al., “Transient adenoviral N-methylpurine DNA glycosylase overexpression imparts chemotherapeutic sensitivity to human breast cancer cells,” Mol Cancer Ther; 3(8):955-67, 2004.
Rogakou et al., “DNA double-stranded breaks induce histone H2AX phosphorylation on serine 139,” Journal of Biological Chemistry, 273:5858-5868, 1998.
Roth and Samson, “3-Methyladenine DNA glycosylase-deficient Aag null mice display unexpected bone marrow alkylation resistance,” Cancer Research, 62:656-660, 2002.
Sambrook, J. and Russell, D. “Molecular Cloning: A Laboratory Manual” (Third Edition), New York: Cold Spring Harbor Laboratory Press, 2000.
Schultz et al., “p53 binding protein 1 (53BP1) is an early participant in the cellular response to DNA double-strand breaks,” Journal of Cell Biology, 151:1381-1390, 2000.
Shiloh, “ATM and related protein kinases: safeguarding genome integrity,” Nature Reviews in Cancer, 3:155-168, 2003.
Silva et al., “RNA-interference-based functional genomics in mammalian cells: reverse genetics coming of age,” Oncogene, 23:8401-8409, 2004.
Sobol and Wilson, “Mammalian DNA B-polymerase in base excision repair of alkylation damage,” Progress in Nucleic Acid Research and Molecular Biology, 68:57-74, 2001.
Sobol et al., “Base Excision Repair Intermediates Induce p53-independent Cytotoxic and Genotoxic Responses,” Journal of Biological Chemistry, 278: 39951-39959, 2003.
Sobol et al., “Mutations associated with base excision repair deficiency and methylation-induced genotoxic stress,” Proceedings of the National Academy of Sciences (USA), 99:6860-6865, 2002.
Sobol et al., “Regulated over-expression of DNA polymerase β mediates early onset cataract in mice,” DNA Repair; 2:609-22, 2003
Sobol et al., “Requirement of mammalian DNA polymerase-B in base-excision repair,” Nature, 379:183-186, 1996.
Sobol et al., “The lyase activity of the DNA repair protein B-polymerase protects from DNA-damage-induced cytotoxicity,” Nature, 405:807-810, 2000.
Sobol et al., “The Role of Base Excision Repair in the Sensitivity and Resistance to Temozolomide-Mediated Cell Death,” Cancer Res 2005; 65: (14):6394-6400, 2005.
Tentori et al., “Systemic administration of GPI 15427, a novel poly(ADP-ribose) polymerase-1 inhibitor, increases the antitumor activity of temozolomide against intracranial melanoma, glioma, lymphoma,” Clinical Cancer Research, 9:5370-5379, 2003.
Tentori, and Graziani, “Pharmacological strategies to increase the antitumor activity of methylating agents,” Current Medical Chemistry, 9:1285-1301, 2002.
Tentori, et al., “Systemic administration of GPI 15427, a novel poly(ADP-ribose) polymerase-1 inhibitor, increases the antitumor activity of temozolomide against intracranial melanoma, glioma, lymphoma,” Clinical Cancer Research; 9(14):5370-9, 2003.
Trivedi et al., “The Role of Base Excision Repair in the Sensitivity and Resistance to Temozolomide Mediated Cell Death,” Cancer Res; 65(14):6394-400, 2005.
Tserng et al., “Pharmacokinetics of 06-benzylguanine (NSC637037) and its metabolite, 8-oxo-06-benzylguanine,” Journal of Clinical Pharmacology, 43:881-893, 2003.
Vogelstein et al., “Surfing the p53 network,” Nature, 408:307-310, 2000.
Ward et al., “Histone H2AX is phosphorylated in an ATR-dependent manner in response to replicational stress,” Journal of Biological Chemistry, 276:47759-47762, 2001.
Wilson et al., “DNA Polymerase B and Mammalian Base Excision Repair,” Cold Spring Harbor, Symposia on Quantitative Biology, 65:143-155, 2001.
Wilson, “Mammalian base excision repair and DNA polymerase B,” Mutation Research, 407:203-215, 1998.
Wood et al., “Human DNA repair genes,” Science, 291-1284-1289, 2001.
Zhou et al. “A role for p53 in base excision repair,” EMBO Journal, 20: 914-923, 2001.

1. A method of killing or retarding the proliferation of one or more neoplastic cells, comprising:
(a) administering a chemotherapeutic agent, radiation, or a combination of a chemotherapeutic agent and radiation to the neoplastic cells; and
(b) administering an inhibitor of DNA polymerase beta to the neoplastic cells in an amount sufficient to reduce the activity of DNA polymerase beta within one or more of said neoplastic cells, whereby the inhibition of DNA polymerase beta in the one or more neoplastic cells potentiates the activity of said chemotherapeutic agent, radiation or a combination of a chemotherapeutic agent and radiation; thereby killing or retarding the proliferation of the neoplastic cell.

2. The method of claim 1, wherein the one or more neoplastic cells are breast cancer cells.

3. The method of claim 1, wherein the inhibitor of DNA polymerase beta is a small inhibitory RNA (siRNA) or a small hairpin RNA (shRNA).

4. The method of claim 3, comprising administering to the cell a vector that expresses the siRNA or the shRNA.

5. The method of claim 4, wherein the vector is a lentiviral vector.

6. The method of claim 3, wherein the shRNA is specific for the 3′ untranslated region of DNA polymerase beta set forth as SEQ ID NO: 5.

7. The method of claim 6, wherein the shRNA is specific for nucleotides 36-54 of SEQ ID NO: 5.

8. The method of claim 1, wherein the method comprises administering to the one or more neoplastic cells a chemotherapeutic agent, and wherein the chemotherapeutic agent is an alkylating agent.

9. The method of claim 7, wherein the alkylating agent is temozolomide.

10. The method of claim 1, wherein said cell is in vitro.

11. The method of claim 1, wherein said cell is in vivo.

12. A method of treating cancer in a subject, comprising:
a) administering to the subject a therapeutically effective amount of a chemotherapeutic agent, radiation, or a combination of a chemotherapeutic agent and radiation; and
(b) administering to the subject an effective amount of an inhibitor of DNA polymerase beta in an amount and at a location sufficient to reduce the activity of DNA polymerase beta within a cancerous cell of the cancer, whereby the inhibition of DNA polymerase beta potentiates the activity of said chemotherapeutic agent
thereby treating the cancer in the subject.

13. The method of claim 12, comprising administering the inhibitor of DNA polymerase beta by intratumoral injection.

14. The method of 12, wherein the inhibitor of DNA polymerase beta is a small inhibitory RNA (siRNA) or a small hairpin RNA (shRNA).

15. The method of claim 12, wherein the cancer is a breast cancer

16. The method of claim 12, comprising administering to the subject a vector encoding the siRNA or the shRNA.

17. The method of claim 16, wherein the shRNA is specific for the 3′ untranslated region of DNA polymerase beta set forth as SEQ ID NO: 5.

18. The method of claim 17, wherein the shRNA is specific for nucleotides 36-54 of SEQ ID NO: 5.

19. The method of claim 16, wherein the vector is a lentiviral vector.

20. The method of claim 12, wherein the method comprises administering a chemotherapeutic agent, and wherein the chemotherapeutic agent is an alkylating agent.

21. The method of claim 20, wherein the alkylating agent is temozolomide.

22. A method of decreasing the growth of breast cancer cells in a subject, comprising:
(a) administering to the subject a therapeutically effective amount of a temozolomide; and
(b) administering to the subject an effective amount of a lentiviral vector that directs expression of a small hairpin mRNA that is specific for nucleotides 36-54 of SEQ ID NO: 5, wherein the shRNA inhibits the expression of DNA polymerase beta, and wherein the lentiviral vector is administered intratumorally
thereby retarding the growth of breast cancer cells in the subject.
